(19) United States
(12) Patent Application Publication
(10) Pub. No.: US 2025/0099363 A1
(43) Pub. Date: Mar. 27, 2025

(54) COSMETIC WATER-IN-OIL EMULSION COMPRISING A SPECIFIC WATER-SOLUBLE UV SCREENING AGENT, A BASE, A VOLATILE OIL, A HYDROPHOBIC FILM-FORMING POLYMER, A POLYOXYALKYLENATED LINEAR POLYDIMETHYLMETHYLSILOXANE AND A VITAMIN B3

(71) Applicant: L'OREAL, Paris (FR)

(72) Inventors: Sonia EYRAUD, Chevilly Larue (FR); Marina MELNIK, Chevilly Larue (FR); Géraldine DEJEAN, Chevilly Larue (FR)

(73) Assignee: L'OREAL, Paris (FR)

(21) Appl. No.: 18/713,218

(22) PCT Filed: Nov. 18, 2022
(86) PCT No.: PCT/EP2022/082417

§ 371 Date: May 24, 2024

(30) Foreign Application Priority Data
Nov. 26, 2021 (FR) FR2112584

(51) Int. Cl. A61K 8/894; A61K 8/06; A61K 8/31; A61K 8/46; A61K 8/49; A61K 8/67; A61Q 1/02

(52) U.S. Cl. CPC A61K 8/894; A61K 8/064; A61K 8/31; A61K 8/466; A61K 8/4946; A61K 8/675; A61Q 1/02; A61K 2800/43

(57) ABSTRACT

Cosmetic water-in-oil emulsion comprising a specific water-soluble UV screening agent, a base, a volatile oil, a hydrophobic film-forming polymer, a polyoxyalkylenated linear poly dimethylmethylsiloxane and a vitamin B3. The present invention relates to a composition in the form of a water-in-oil emulsion, in particular comprising a physiologically acceptable medium, especially for coating keratin materials, more particularly for making up and/or caring for keratin materials, comprising: a) at least one oily continuous phase comprising at least one volatile oil chosen from volatile hydrocarbon oils, volatile silicone oils and their mixtures, and b) at least one aqueous phase dispersed in said oily phase, comprising at least one water-soluble organic UV screening agent comprising at least one benzylidene camphorsulfonic acid group and/or at least one water-soluble organic UV screening agent comprising at least one benzoxazole sulfonic acid group, and c) at least one organic or inorganic base capable of partially or completely neutralizing said water-soluble organic UV screening agent(s), and d) at least one hydrophobic film-forming polymer, and c) at least one emulsifying surfactant chosen from polyoxyalkylenated linear poly dimethylmethylsiloxanes with an HLB≤8.0, and f) at least one vitamin B3; said composition not containing a nonvolatile ester oil.

### TECHNICAL FIELD

The present invention relates to the field of caring for and/or making up keratin materials and is targeted at providing compositions more particularly dedicated to caring for and/or making up the skin.

The skin is not a smooth surface of uniform color and exhibits reliefs and microreliefs, such as pores, fine lines, wrinkles, spots, scars and dry areas, which form a somewhat bumpy surface. Quite often, this surface, with its irregularities, forms a pleasant-looking whole but the irregularities are such that sometimes the surface is considered unattractive.

Cosmetic makeup and/or care compositions are commonly employed for hiding, smoothing out and/or unifying imperfections of the relief of the skin, such as pores, wrinkles and/or fine lines and/or scars. In this regard, numerous solid or fluid, anhydrous or non-anhydrous, formulations have been developed to date.

The application of a makeup composition, such as a foundation, is the most effective approach for enhancing the beauty of irregular skin, by making it possible to hide stains and dyschromias, to reduce the visibility of relief imperfections, such as pores and wrinkles, and to conceal spots and acne marks; in this regard, coverage is one of the main properties sought. Use is generally made, in these compositions, at high amounts, of pigments based on metal oxides, such as iron oxides and titanium oxides, in combination with particles known as fillers having a soft focus effect. However, these compositions have a tendency to accumulate in the reliefs, such as pores and wrinkles, resulting in a nonuniform deposit on the skin highlighting the imperfections thereof and giving a matt finish perceived as unnatural on the skin.

There thus remains a need to find novel cosmetic compositions which are stable on storage, in particular stable with respect to temperature variations, and which make it possible to efficiently smooth out and conceal the imperfections of the skin by virtue of a good coverage performance, which lasts throughout the day (wear property), and to substantially reduce, indeed even to suppress, the effects of highlighting the reliefs and concealing effect on keratin substances, such as the skin, to obtain good uniformity of the complexion and preferably a less matt, more natural, appearance, and also cosmetic properties satisfactory for the comfort of the consumer.

In the course of its research, the Applicant Company has discovered, surprisingly, that this objective can be achieved with a composition in the form of a water-in-oil emulsion comprising:
a) at least one oily continuous phase comprising at least one volatile oil chosen from volatile hydrocarbon oils, volatile silicone oils and their mixtures, and
b) at least one aqueous phase dispersed in said oily phase, comprising at least one water-soluble organic UV screening agent comprising at least one benzylidenecamphor sulfonic acid group and/or at least one water-soluble organic UV screening agent comprising at least one benzoxazole sulfonic acid group, and
c) at least one organic or inorganic base capable of partially or completely neutralizing said water-soluble organic UV screening agent(s), and
d) at least one hydrophobic film-forming polymer, and
e) at least one emulsifying surfactant chosen from polyoxyalkylenated linear polydimethylmethylsiloxanes with an HLB≤8.0, and
f) at least one vitamin B3;
said composition not containing a nonvolatile ester oil.

This discovery forms the basis of the invention.

The present invention relates to a composition in the form of a water-in-oil emulsion, in particular comprising a physiologically acceptable medium, especially for coating keratin materials, more particularly for making up and/or caring for keratin materials, comprising:
a) at least one oily continuous phase comprising at least one volatile oil chosen from volatile hydrocarbon oils, volatile silicone oils and their mixtures, and
b) at least one aqueous phase dispersed in said oily phase, comprising at least one water-soluble organic UV screening agent comprising at least one benzylidenecamphor sulfonic acid group and/or at least one water-soluble organic UV screening agent comprising at least one benzoxazole sulfonic acid group, and
c) at least one organic or inorganic base capable of partially or completely neutralizing said water-soluble organic UV screening agent(s), and
d) at least one hydrophobic film-forming polymer, and
e) at least one emulsifying surfactant chosen from polyoxyalkylenated linear polydimethylmethylsiloxanes with an HLB≤8.0, and
f) at least one vitamin B3;
said composition not containing a nonvolatile ester oil.

The invention also relates to a process for coating keratin materials, more particularly for making up and/or caring for keratin materials, such as the skin, characterized in that it comprises the application to keratin materials of an emulsion as defined above.

The invention more particularly relates to a process for making up and/or caring for the skin, characterized in that it comprises the application to the skin of an emulsion as defined above.

### Definitions

In the context of the present invention, the term “keratin materials” means the skin and more particularly the areas such as the face, the cheeks, the hands, the body, the legs and thighs, the area around the eyes, and the eyelids.

The term “physiologically acceptable” is understood to mean compatible with the skin and/or its superficial body growths, which exhibits a pleasant color, odor and feel and which does not cause unacceptable discomfort (stinging or tautness) liable to dissuade the consumer from using this composition.

Within the meaning of the present invention, the term “emulsifying surfactant” is understood to mean an amphiphilic surfactant compound, that is to say one which exhibits two parts of different polarity. In general, one is lipophilic (soluble or dispersible in an oily phase) and the other is hydrophilic (soluble or dispersible in water). Emulsifying surfactants are characterized by the value of their HLB (Hydrophilic Lipophilic Balance), the HLB being the ratio of the hydrophilic part to the lipophilic part in the molecule. The term “HLB” is well known to a person skilled in the art and is described, for example, in The HLB System. A Time-Saving Guide to Emulsifier Selection (published by ICI Americas Inc.; 1984). For emulsifying surfactants, the HLB generally ranges from 3 to 8 for the preparation of W/O emulsions. The HLB of the surfactant(s) used according to the invention can be determined by the Griffin method or the Davies method.

The term “water-in-oil emulsion” is understood to mean a composition comprising an oily phase and an aqueous phase which are immiscible; the aqueous phase being dispersed in the form of droplets in the oily phase (described as continuous) so as to obtain a macroscopically homogeneous composition.

The term “oil” is understood to mean a fatty organic substance which is liquid at ambient temperature (25° C.) and atmospheric pressure (760 mmHg, i.e. 105 Pa).

The term “nonvolatile oil” is understood to mean an oil which remains on the keratin material at ambient temperature (25° C.) and atmospheric pressure (760 mmHg) for at least several hours and which has in particular a vapor pressure of less than 10⁻³ mmHg (0.13 Pa).

The term “nonvolatile ester oil” is understood to mean any nonvolatile oil constituted of a single ester compound or of a mixture of ester compounds. Said ester oil or the mixture of ester oils is in particular constituted of one or more mono-, di- or triester(s) of a carboxylic acid having from 12 to 30 carbon atoms and of a monoalcohol or of a polyol, such as a glycol, in particular an alkylene glycol, for example the mixtures of esters with the INCI name: Propylene Glycol Dicaprylate/Dicaprate and Butylene Glycol Dicaprylate/Dicaprate.

The term “composition not containing a nonvolatile ester oil” is understood to mean any composition comprising less than 2.0% by weight of nonvolatile ester oil, indeed even less than 1.0% by weight, indeed even less than 0.5% by weight, with respect to the total weight of the composition, indeed even devoid of nonvolatile ester oil.

### Oily Continuous Phase

The composition of the invention comprises an oily continuous phase comprising at least one volatile oil chosen from volatile hydrocarbon oils, volatile silicone oils and their mixtures.

Said phase is liquid (in the absence of structuring agent) at ambient temperature (25° C.) and atmospheric pressure (760 mmHg). It is organic, namely comprising at least carbon and hydrogen atoms, and water-immiscible.

The oily phase comprises at least one volatile oil and optionally ingredients which are soluble or miscible in said phase.

The term “oil” is understood to mean a fatty substance which is liquid at ambient temperature (25° C.) and atmospheric pressure (760 mmHg, i.e. 105 Pa).

The total concentration of oily phase of the composition of the invention preferably varies from 20% to 95% by weight and more particularly ranges from 30% to 60% by weight, with respect to the total weight of the composition.

### Volatile Oils

Within the meaning of the invention, the term “volatile oil” is understood to mean any oil capable of evaporating on contact with the skin in less than one hour, at ambient temperature (25° C.) and atmospheric pressure (760 mmHg). The volatile oil is a volatile cosmetic compound, which is liquid at ambient temperature, having in particular a non-zero vapor pressure, at ambient temperature and atmospheric pressure, in particular having a vapor pressure ranging from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile oil or oils are preferably present at a concentration from 5% to 45% by weight, more preferentially ranging from 10% to 40% by weight and more preferentially still ranging from 12% to 35% by weight, with respect to the total weight of the composition.

### a) Volatile Hydrocarbon Oils

The term “hydrocarbon oil” is understood to mean an oil comprising mainly carbon and hydrogen atoms and optionally one or more functional groups chosen from hydroxyl, ester, ether or carboxyl functional groups

Mention may be made, as examples of volatile hydrocarbon oil which can be used in the invention, of hydrocarbon oils having from 8 to 16 carbon atoms, and in particular C₈-C₁₆ isoalkanes of petroleum origin (also known as isoparaffins), such as isododecane (also known as 2,2,4,4,6-pentamethylheptane) and isohexadecane, for example the oils sold under the trade names Isopar® or Permethyl®, branched C₈-C₁₆ esters, isohexyl neopentanoate and their mixtures. Other volatile hydrocarbon oils, such as petroleum distillates, in particular those sold under the name Shell Solt by Shell, can also be used; volatile linear alkanes, such as those described in the patent application DE10 2008 012 457 from Cognis.

Use will more particularly be made of isododecane.

### b) Volatile Silicone Oils

Within the meaning of the present invention, the term “silicone oil” is understood to mean an oil comprising at least one silicon atom, and in particular at least one Si—O group, and more particularly an organopolysiloxane.

Mention may be made, among the volatile silicone oils which can be used in the compositions, for example, of volatile linear or cyclic silicone oils, those having a viscosity≤8 centistokes (8×10⁻⁶ m²/s) and having in particular from 2 to 7 silicon atoms, these silicones optionally comprising alkyl or alkoxy groups having from 1 to 10 carbon atoms.

Use will more preferentially be made, among these oils, of the volatile linear silicone oils with a viscosity ranging from 1 to 8 cSt (from 1×10⁻⁶ to 8×10⁻⁶ m²/s), such as
octamethyltrisiloxane, in particular sold under the name Xiameter PMX-200 Silicone Fluid 1CS® by Dow Corning;
decamethyltetrasiloxane, in particular sold under the name Xiameter PMX-200 Silicone Fluid 1.5CS® by Dow Corning;
dodecamethylpentasiloxane, such as the commercial products sold under the names KF-96L-2CS® and DM-Fluid-2CS® by Shin Etsu, Berb-DM2® by BRB International or Xiameter PMX-200 Silicone Fluid 2CS® by Dow Corning;
their mixtures.

Use will more particularly be made of dodecamethylpentasiloxane.

Mention may be made, as volatile cyclic silicone oil which can be used in the invention, of octamethylcyclotetrasiloxane, decamethylcyclopentasiloxane, dodecamethylcyclohexasiloxane and their mixtures.

According to a specific form of the invention, use will be made of a mixture of at least one volatile hydrocarbon oil and of at least one volatile silicone oil, and more particularly of a mixture of isododecane and of dodecamethylpentasiloxane.

### Aqueous Phase

The aqueous phase comprises water and optionally water-soluble or water-miscible ingredients, such as water-soluble solvents.

A water suitable for the invention may be a floral water, such as cornflower water, and/or a mineral water, such as Vittel water, Lucas water or La Roche-Posay water, and/or a thermal water.

In the present invention, the term “water-soluble solvent” denotes a compound which is liquid at ambient temperature and water-miscible (miscibility in water of greater than 50% by weight at 25° C. and atmospheric pressure).

The water-soluble solvents which can be used in the composition of the invention can in addition be volatile.

Mention may in particular be made, among the water-soluble solvents which can be used in the composition in accordance with the invention, of lower monoalcohols having from 1 to 5 carbon atoms, such as ethanol and isopropanol, glycols having from 2 to 8 carbon atoms, such as ethylene glycol, propylene glycol, 1,3-butylene glycol, propanediol, pentylene glycol, glycerol and dipropylene glycol, C₃-C₄ ketones and C₂-C₄ aldehydes.

The aqueous phase is preferably present in a concentration of at least 20% by weight, preferably ranging from 30% to 60% by weight, more particularly from 35% to 50% by weight, with respect to the total weight of said composition.

### Water-Soluble Organic UV Screening Agent

The compositions according to the invention comprise at least one water-soluble organic UV screening agent chosen from UV screening agents comprising at least one benzylidenecamphor sulfonic acid group, UV screening agents comprising at least one benzoxazole sulfonic acid group and their mixtures.

The term “water-soluble organic UV screening agent” is understood to mean any organic compound which screens out UV radiation in the wavelength range 280 to 400 nm capable of being completely dissolved in the molecular state or miscible in a liquid aqueous phase or else of being dissolved in colloidal form (for example in micellar form) in a liquid aqueous phase.

### a) Water-Soluble UV Screening Agents Having Benzylidenecamphor Sulfonic Acid Group(s)

Mention may be made, among the water-soluble organic UV screening agents having benzylidenecamphor sulfonic acid group(s), of benzene-1,4-di(3-methylidene-10-camphorsulfonic acid) with the INCI name: Terephthalylidene Dicamphor Sulfonic Acid and its various salts, described in particular in the patent applications FR-A-2 528 420 and FR-A-2 639 347.

These water-soluble UV screening agents correspond to the following general formula (I)

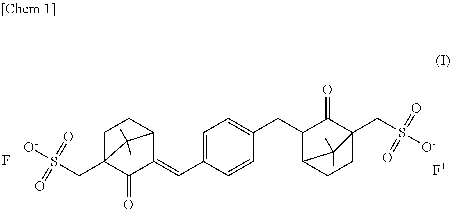

in which F denotes a hydrogen atom, an alkali metal or also an NH(R¹)³⁺ radical in which the R¹ radicals, which can be identical or different, denote a hydrogen atom or a C₁-C₄ alkyl or hydroxyalkyl radical or also an Mⁿ⁺ group denoting a polyvalent metal cation in which n is equal to 2 or 3 or 4, preferably a metal cation chosen from Ca²⁺, Zn²⁺, Mg²⁺, Ba²⁺, Al³⁺ and Zr⁴⁺. It is clearly understood that the compounds of formula (I) above can give rise to the “cis-trans” isomer around one or more double bond(s) and that all the isomers come within the context of the present invention.

Mention may also be made of the following UV screening agents:
Benzylidene Camphor Sulfonic Acid, manufactured under the name Mexoryl SL® by Chimex,
Camphor Benzalkonium Methosulfate, manufactured under the name Mexoryl SO® by Chimex,
Polyacrylamidomethyl Benzylidene Camphor, manufactured under the name Mexoryl SW® by Chimex.

### b) Water-Soluble UV Screening Agents Having Benzoxazole Sulfonic Acid Group(s)

Mention may be made, among the water-soluble organic UV screening agents having benzoxazole sulfonic acid group(s) and their salts according to the present invention, of the compounds comprising at least two benzoxazole sulfonic acid groups, such as those described in the patent application EP-A-0 669 323. They are described and prepared according to the syntheses indicated in the patent U.S. Pat. No. 2,463,264 and also the patent application EP 0 669 323 B1.

Mention may be made, among these compounds, of 1,4-bis-benzimidazolyl-phenylene-3,3′,5,5′-tetrasulfonic acid with the following structure:

It is sold in the form of the disodium salt with the INCI name: Disodium Phenyl Dibenzimidazole Tetrasulfonate and under the name Neoheliopan AP® by Symrise having the following formula:

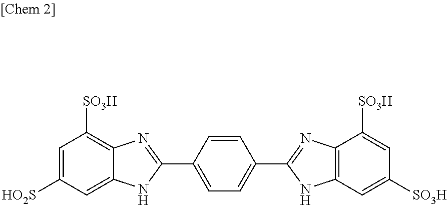

Mention may also be made, among water-soluble organic UV screening agents having benzoxazole sulfonic acid group(s) and their salts according to the present invention, of the UV screening agent with the INCI name Phenylbenzimidazole Sulfonic Acid, sold in particular under the trade name Eusolex 232® by Merck.

Use will preferably be made of a water-soluble UV screening agent chosen from Terephthalylidene Dicamphor Sulfonic Acid, 1,4-bis-benzimidazolyl-phenylene-3,3′,5,5′-tetrasulfonic acid, Phenylbenzimidazole Sulfonic Acid and their mixtures, and more particularly Phenylbenzimidazole Sulfonic Acid.

The water-soluble organic UV screening agent(s) in accordance with the invention are partially or completely neutralized by an inorganic base.

The water-soluble UV screening agent(s) in accordance with the invention are preferably present in the composition of the invention at concentrations ranging from 0.1% to 10% by weight, more preferentially ranging from 1% to 8% by weight and more preferentially still ranging from 2% to 5% by weight, with respect to the total weight of the composition.

### Base

The compositions according to the invention comprise at least one organic or inorganic base capable of partially or completely neutralizing said water-soluble organic UV screening agent(s) of the invention.

The term “organic base” is understood to mean a hydrocarbon molecule comprising at least carbon atoms, hydrogen atoms and at least one heteroatom, such as oxygen, nitrogen or sulfur, capable of capturing one or more protons in an aqueous medium.

The term “inorganic base” is understood to mean a molecule not comprising a carbon atom capable of capturing one or more protons in an aqueous medium.

The base of organic origin is preferably chosen from amino acids, such as arginine; alkanolamines, such as monoethanolamine, diethanolamine, triethanolamine, triisopropanolamine or aminomethyl propanol; primary (poly) hydroxyalkylamines, such as 2-amino-2-(hydroxymethyl) propane-1,3-diol (also known as tromethamine) and aminomethylpropanediol; and their mixtures.

The inorganic base in accordance with the invention is preferably chosen from alkali metal cation bases, such as sodium hydroxide (NaOH), potassium hydroxide (KOH), lithium hydroxide and cesium hydroxide, or alkaline earth metal cation bases, such as magnesium hydroxide, calcium hydroxide or barium hydroxide.

Use will in particular be made of an inorganic base, and more particularly sodium hydroxide.

### Hydrophobic Film-Forming Polymer

Within the meaning of the invention, the term “polymer” is understood to mean a compound corresponding to the repetition of one or more units (these units resulting from compounds known as monomers). This or these unit(s) are repeated at least twice and preferably at least three times.

Within the meaning of the present invention, the term “hydrophobic film-forming polymer” is understood to denote a film-forming polymer which is devoid of affinity for water and, as such, which does not lend itself to a formulation in the form of a solute in an aqueous medium. In particular, the term “hydrophobic polymer” is understood to mean a polymer having a solubility in water at 25° C. of less than 1% by weight.

The term “film-forming polymer” is understood to mean a polymer capable of forming, by itself alone or in the presence of an auxiliary film-forming agent, a macroscopically continuous film on a support, in particular on keratin materials, preferably a cohesive film, and better still a film, the cohesion and the mechanical properties of which are such that said film can be isolable and manipulable in isolation, for example when said film is prepared by pouring onto a non-stick surface, such as a Teflon-coated or silicone-coated surface.

In particular, the hydrophobic film-forming polymer is a polymer chosen from the group consisting of film-forming polymers which are soluble in an organic solvent medium, in particular fat-soluble polymers; this means that the polymer is soluble or miscible in the organic medium and will form a single homogeneous phase when it is incorporated in the medium.

Mention may in particular be made, as hydrophobic film-forming polymer, of:
silicone resins;
block ethylenic copolymers;
vinyl polymers comprising at least one unit derived from carbosiloxane dendrimer;
silicone-acrylate copolymers;
their mixtures.

Preferentially, a composition according to the invention comprises from 0.5% to 15% by weight, more preferentially from 1% to 10% by weight, more particularly from 2% to 7% by weight, as active material of hydrophobic film-forming polymer(s), with respect to the total weight of the composition.

### I. Silicone Resins

More generally, the term “resin” is understood to mean a compound, the structure of which is three-dimensional. “Silicone resins” are also referred to as “siloxane resins”. Thus, within the meaning of the present invention, a polydimethylsiloxane is not a silicone resin.

The nomenclature of silicone resins (also referred to as siloxane resins) is known under the name “MDTQ”, the resin being described as a function of the various siloxane monomer units which it comprises, each of the letters “MDTQ” characterizing one type of unit.

The letter M represents the Monofunctional unit of formula R₁R₂R₃SiO_{1/2}, the silicon atom being connected to only one oxygen atom in the polymer comprising this unit.

The letter D signifies a Difunctional unit R₁R₂SiO_{2/2} in which the silicon atom is connected to two oxygen atoms.

The letter T represents a Trifunctional unit of formula R₁SiO_{3/2}.

Such resins are described, for example, in the Encyclopedia of Polymer Science and Engineering, Vol. 15, John Wiley & Sons, New York, (1989), pp. 265-270, and U.S. Pat. Nos. 2,676,182, 3,627,851, 3,772,247, 5,248,739 or U.S. Pat. Nos. 5,082,706, 5,319,040, 5,302,685 and 4,935,484.

In the M, D and T units defined above, R, namely R₁ and R₂, represents a hydrocarbon radical (in particular an alkyl radical) having from 1 to 10 carbon atoms, a phenyl group, a phenylalkyl group or else a hydroxyl group.

Finally, the letter Q signifies a tetrafunctional unit SiO_{4/2} in which the silicon atom is bonded to four oxygen atoms, which are themselves bonded to the remainder of the polymer.

Various silicone resins with different properties can be obtained from these different units, the properties of these polymers varying as a function of the type of monomer (or units), of the nature and number of the R radical, of the length of the polymer chain, of the degree of branching and of the size of the pendent chains.

Use may be made, as silicone resins which can be used in the compositions according to the invention, for example, of silicone resins of MQ type, of T type or of MQT type.

Mention may be made, as examples of silicone resins of MQ type, of the alkylsiloxysilicates of formula [(R₁)₃SiO_{1/2}]ₓ(SiO_{4/2})_{y} (MQ units) in which x and y are integers ranging from 50 to 80, and such that the R₁ group represents a radical as defined above and is preferably an alkyl group having from 1 to 8 carbon atoms or a hydroxyl group, preferably a methyl group.

Mention may be made, as examples of solid silicone resins of MQ type of trimethylsiloxysilicate type, of those sold under the reference SR1000® by General Electric, under the reference TMS 803® by Wacker and under the names KF-7312®J by Shin-Etsu and DC 749® and DC 593® by Dow Corning.

Mention may also be made, as silicone resins comprising siloxysilicate MQ units, of phenylalkylsiloxysilicate resins, such as phenylpropyldimethylsiloxysilicate (Silshine 151® sold by General Electric). The preparation of such resins is described in particular in the patent U.S. Pat. No. 5,817,302.

### a) T Resins

Mention may be made, as examples of silicone resins of T type, of the polysilsesquioxanes of formula (RSiO_{3/2})ₓ (T units) in which x is greater than 100 and such that the R group is an alkyl group having from 1 to 10 carbon atoms, it being possible for said polysilsesquioxanes to additionally comprise Si—OH end groups.

Preferably, use may be made of polymethylsilsesquioxane resins in which R represents a methyl group, such as, for example, those sold:
by Wacker under the reference Resin MK®, such as Belsil PMS MK®: polymer comprising CH₃SiO_{3/2} repeat units (T units), which can also comprise up to 1% by weight of (CH₃)₂SiO_{2/2} units (D units) and exhibiting an average molecular weight of approximately 10 000 g/mol, or
by Shin-Etsu under the reference KR-220L®, which are composed of T units of formula CH₃SiO_{3/2} and have Si—OH (silanol) end groups, under the reference KR-242AR, which comprise 98% of T units and 2% of D dimethyl units and have Si—OH end groups, or also under the reference KR-251®, comprising 88% of T units and 12% of D dimethyl units and have Si—OH end groups.

### b) MQT Resins

Resins comprising MQT units which are in particular known are those mentioned in the document U.S. Pat. No. 5,110,890.

A preferred form of resins of MQT type are MQT-propyl (also known as MQTPr) resins. Such resins which can be used in the compositions according to the invention are in particular those described and prepared in the application WO 2005/075542.

The MQT-propyl resin preferably comprises the following units:
(i) (R1₃SiO_{1/2})ₐ;
(ii) (R2₂SiO_{2/2})_{b};
(iii) (R3SiO_{3/2})_{c}; and
(iv) (SiO_{4/2})_{d};
with:
   R1, R2 and R3 independently representing a hydrocarbon (in particular alkyl) radical having from 1 to 10 carbon atoms, a phenyl group, a phenylalkyl group or else a hydroxyl group and preferably an alkyl radical having from 1 to 8 carbon atoms or a phenyl group,
   a being of between 0.05 and 0.5, -b being of between zero and 0.3, -c being greater than zero,
   d being of between 0.05 and 0.6,
   a+b+c+d=1, and a, b, c and d being molar fractions,
provided that more than 40 mol % of the R3 groups of the siloxane resin are propyl groups.

Preferably, the siloxane resin comprises the following units:
(i) (R1₃SiO_{1/2}) a;
(iii) (R3SiO_{3/2})_{c}; and
(iv) (SiO_{4/2})_{d};
with:
   R1 and R3 independently representing an alkyl group having from 1 to 8 carbon atoms, R1 preferably being a methyl group and R3 preferably being a propyl group,
   a being of between 0.05 and 0.5 and preferably between 0.15 and 0.4,
   c being greater than zero, preferably between 0.15 and 0.4,
   d being of between 0.05 and 0.6, preferably between 0.2 and 0.6 or also between 0.2 and 0.55,
   a+b+c+d=1 and a, b, c and d being molar fractions, provided that more than 40 mol % of the R3 groups of the siloxane resin are propyl groups.

The siloxane resins which can be used according to the invention can be obtained by a process comprising the reaction of:
A) an MQ resin comprising at least 80 mol % of (R1₃SiO_{1/2})ₐ and (SiO_{4/2})_{d} units; with
   R1 representing an alkyl group having from 1 to 8 carbon atoms, an aryl group, a carbinol group or an amino group,
   a and d being greater than zero,
   the ratio a/d being of between 0.5 and 1.5;
and of:
B) a T-propyl resin comprising at least 80 mol % of (R3SiO_{3/2})_{c} units; with
   R3 representing an alkyl group having from 1 to 8 carbon atoms, an aryl group, a carbinol group or an amino group,
   c being greater than zero, provided that at least 40 mol % of the R3 groups are propyl groups, where the ratio by weight A/B is of between 95:5 and 15:85 and preferably the ratio by weight A/B is 30:70.

Advantageously, the ratio by weight A/B is of between 95:5 and 15:85. Preferably, the ratio A/B is less than or equal to 70:30. These preferred ratios have proved to make possible comfortable deposits due to the absence of percolation of the rigid particles of MQ resin in the deposit.

Thus, preferably, the silicone resin is chosen from resins of MQ type, chosen in particular from (i) alkylsiloxysilicates, which can be trimethylsiloxysilicates, of formula [R1₃SiO_{1/2}]ₓ(SiO_{4/2})_{y}, in which x and y are integers ranging from 50 to 80, and such that the R1 group represents a hydrocarbon radical having from 1 to 10 carbon atoms, a phenyl group, a phenylalkyl group or else a hydroxyl group, and preferably is an alkyl group having from 1 to 8 carbon atoms, preferably a methyl group, and (ii) phenylalkylsiloxysilicate resins, such as phenylpropyldimethylsiloxysilicate resin.

Advantageously, a composition according to the invention comprises, as hydrophobic film-forming polymer, at least one trimethylsiloxysilicate resin, such as those sold under the reference Silsoft 74 by Momentive Performance Materials, SR1000@ by General Electric, under the reference TMS 803® by Wacker and under the names KF-7312RJ by Shin-Etsu and DC 749® and DC 593® by Dow Corning.

### c) Silsesquioxane Resins

Mention may be made, among the silsesquioxane resins which can be used in the compositions in accordance with the invention, of the alkylsilsesquioxane resins which are silsesquioxane homopolymers and/or copolymers having an average siloxane unit of formula R1ₙSiO_{(4-n)/2}, where each R1 independently denotes a hydrogen atom or a C₁-C₁₀ alkyl group, where more than 80 mol % of the R1 radicals represent a C₃-C₁₀ alkyl group, and n is a number from 1.0 to 1.4, and more particularly use will be made of a silsesquioxane copolymer in which more than 60 mol % comprises R1SiO_{3/2} units in which R1 has the definition indicated above.

Preferably, the silsesquioxane resin is chosen so that R1 is a C₁-C₁₀ alkyl group, preferably a C₁-C₄ alkyl group and more particularly a propyl group. Use will more particularly be made of a polypropylsilsesquioxane or t-propylsilsesquioxane resin (INCI name: Polypropylsilsesquioxane (and) Isododecane), such as the product sold under the trade name Dow Corning® 670 Fluid by Dow Corning.

### II. Block Ethylenic Copolymers

The hydrophobic film-forming polymer can be a block ethylenic copolymer, containing at least one first block having a glass transition temperature (Tg) of greater than or equal to 40° C. and resulting in all or part from one or more first monomers, which are such that the homopolymer prepared from these monomers has a glass transition temperature of greater than or equal to 40° C., and at least one second block having a glass transition temperature of less than or equal to 20° C. and resulting in all or part from one or more second monomers, which are such that the homopolymer prepared from these monomers has a glass transition temperature of less than or equal to 20° C., said first block and said second block being connected together via a random intermediate segment comprising at least one of said first constituent monomers of the first block and at least one of said second constituent monomers of the second block, and said block copolymer having a polydispersity index I of greater than 2.

Polymers of this type suitable for the invention are described in the document EP 1 411 069.

Mention may more particularly be made, as examples of such polymers, of Mexomere PAS® (acrylic acid/isobutyl acrylate/isobornyl acrylate copolymer 50% diluted in isododecane) sold by Chimex.

The block ethylenic copolymer can in particular be a diblock, triblock, multiblock, radial or star-branched copolymer, or their mixtures, as described in the application US-A-2002/005562 and in the patent U.S. Pat. No. 5,221,534.

The copolymer can exhibit at least one block, the glass transition temperature of which is preferably less than 20° C., preferably less than or equal to 0° C., preferably less than or equal to −20° C. and more preferably less than or equal to −40° C. The glass transition temperature of said block can be of between −150° C. and 20° C. and in particular between −100° C. and 0° C. The copolymer is amorphous, formed by polymerization of an olefin. The olefin can in particular be an elastomeric ethylenically unsaturated monomer.

Mention may be made, as examples of olefins, of ethylenic carbide monomers, having in particular one or two ethylenic unsaturations and having from 2 to 5 carbon atoms, such as ethylene, propylene, butadiene, isoprene or pentadiene.

Advantageously, the hydrocarbon block copolymer is an amorphous block copolymer of styrene and of olefin.

Block copolymers comprising at least one styrene block and at least one block comprising units chosen from butadiene, ethylene, propylene, butylene, isoprene or one of their mixtures are preferred in particular.

According to a preferred embodiment, the hydrocarbon block copolymer is hydrogenated in order to reduce the residual ethylenic unsaturations after the polymerization of the monomers.

In particular, the hydrocarbon block copolymer is an optionally hydrogenated copolymer, having styrene blocks and having ethylene/C₃-C₄ alkylene blocks.

Mention may be made, as diblock copolymers, which are preferably hydrogenated, of styrene-ethylene/propylene copolymers, styrene-ethylene/butadiene copolymers or styrene-ethylene/butylene copolymers. Diblock polymers are sold in particular under the name Kraton® G1701E by Kraton Polymers.

Mention may be made, as triblock copolymers, which are preferably hydrogenated, of styrene-ethylene/propylene-styrene copolymers, styrene-ethylene/butadiene-styrene copolymers, styrene-ethylene/butylene-styrene copolymers, styrene-isoprene-styrene copolymers or styrene-butadiene-styrene copolymers. Triblock polymers are sold in particular under the names Kraton® G1650, Kraton® G1652, Kraton® D1101, Kraton® D1102 or Kraton® D1160 by Kraton Polymers.

According to one embodiment of the present invention, the hydrocarbon block copolymer is a styrene-ethylene/butylene-styrene triblock copolymer.

According to a preferred embodiment of the invention, it is possible in particular to use a mixture of a styrene-butylene/ethylene-styrene triblock copolymer and of a styrene-ethylene/butylene diblock copolymer, in particular those sold under the name Kraton® G1657M by Kraton Polymers.

Use may also be made of a mixture of hydrogenated styrene-butylene/ethylene-styrene triblock copolymer and of hydrogenated ethylene-propylene-styrene star-branched polymer, such a mixture being in particular in isododecane. Such mixtures are sold, for example, by Penreco under the trade names Versagel® M5960 and Versagel® M5670.

III. Vinyl Polymer Comprising at Least One Unit Derived from Carbosiloxane Dendrimer

The hydrophobic film-forming polymer can also be chosen from vinyl polymers comprising at least one unit derived from carbosiloxane dendrimer.

The vinyl polymer(s) have in particular a backbone and at least one side chain, which side chain comprises a unit derived from carbosiloxane dendrimer exhibiting a carbosiloxane dendrimer structure.

In particular, use may be made of vinyl polymers comprising at least one carbosiloxane dendrimer unit as described in the applications WO03/045337 and EP 963 751 from Dow Corning.

In the context of the present invention, the term “carbosiloxane dendrimer structure” represents a molecular structure possessing branched groups having high molecular weights, said structure having high regularity in the radial direction starting from the bond to the backbone. Such carbosiloxane dendrimer structures are described in the form of a highly branched siloxane-silylalkylene copolymer in the laid-open Japanese patent application Kokai 9-171 154.

A vinyl polymer having at least one unit derived from carbosiloxane dendrimer has a side molecular chain containing a carbosiloxane dendrimer structure, and can result from the polymerization:
of 0 to 99.9 parts by weight of a vinyl monomer; and
of 100 to 0.1 parts by weight of a carbosiloxane dendrimer containing a radically polymerizable organic group, represented by the general formula:
   in which Y represents a radically polymerizable organic group,
   R¹ represents an aryl group or an alkyl group having from 1 to 10 carbon atoms, and
   Xⁱ represents a silylalkyl group which, when i=1, is represented by the following formula:
   in which R¹ is as defined above, R² represents an alkylene group having from 2 to 10 carbon atoms, R³ represents an alkyl group having from 1 to 10 carbon atoms, Xⁱ⁺¹ represents a hydrogen atom, an alkyl group having from 1 to 10 carbon atoms, an aryl group or the silylalkyl group defined above with i=i+1, i is an integer from 1 to 10 which represents the generation of said silylalkyl group and aⁱ is an integer from 0 to 3;
   where said radically polymerizable organic group contained in the component (A) is chosen from:
   organic groups containing a methacrylic group or an acrylic group and which are represented by the following formulae; and in which R⁴ represents a hydrogen atom or an alkyl group,
   R⁵ represents an alkylene group having from 1 to 10 carbon atoms and organic groups containing a styryl group and which are represented by the following formula:
   in which R⁶ represents a hydrogen atom or an alkyl group, R⁷ represents an alkyl group having from 1 to 10 carbon atoms, R⁸ represents an alkylene group having from 1 to 10 carbon atoms, b is an integer from 0 to 4 and c has the value 0 or 1, such that, if c is 0,
   —(R⁸)_{c}— represents a bond.

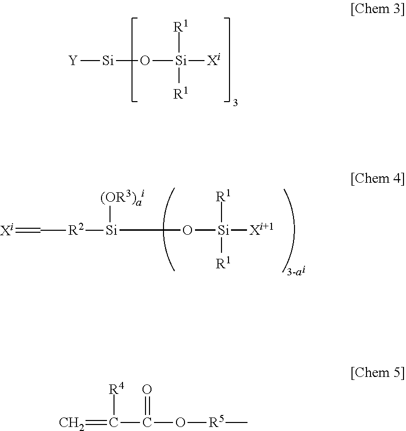

A vinyl polymer having at least one unit derived from carbosiloxane dendrimer has a side molecular chain containing a carbosiloxane dendrimer structure, and can result from the polymerization:
of 0 to 99.9 parts by weight of a vinyl monomer; and
of 100 to 0.1 parts by weight of a carbosiloxane dendrimer containing a radically polymerizable organic group, represented by the general formula:
   in which Y represents a radically polymerizable organic group,
   R¹ represents an aryl group or an alkyl group having from 1 to 10 carbon atoms, and
   Xⁱ represents a silylalkyl group which, when i=1, is represented by the following formula:
   in which R¹ is as defined above, R² represents an alkylene group having from 2 to 10 carbon atoms, R³ represents an alkyl group having from 1 to 10 carbon atoms, Xⁱ⁺¹ represents a hydrogen atom, an alkyl group having from 1 to 10 carbon atoms, an aryl group or the silylalkyl group defined above with i=i+1, i is an integer from 1 to 10 which represents the generation of said silylalkyl group and aⁱ is an integer from 0 to 3;
   where said radically polymerizable organic group contained in the component (A) is chosen from:
   organic groups containing a methacrylic group or an acrylic group and which are represented by the following formulae; and in which R4 represents a hydrogen atom or an alkyl group,
   R⁵ represents an alkylene group having from 1 to 10 carbon atoms and organic groups containing a styryl group and which are represented by the following formula: in which R⁶ represents a hydrogen atom or an alkyl group, R⁷ represents an alkyl group having from 1 to 10 carbon atoms, R⁸ represents an alkylene group having from 1 to 10 carbon atoms, b is an integer from 0 to 4 and c has the value 0 or 1, such that, if c is 0,
   —(R⁸)_{c}— represents a bond.

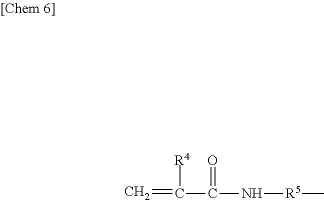

A vinyl polymer having at least one unit derived from carbosiloxane dendrimer has a side molecular chain containing a carbosiloxane dendrimer structure, and can result from the polymerization:
of 0 to 99.9 parts by weight of a vinyl monomer; and
of 100 to 0.1 parts by weight of a carbosiloxane dendrimer containing a radically polymerizable organic group, represented by the general formula:
   in which Y represents a radically polymerizable organic group,
   R¹ represents an aryl group or an alkyl group having from 1 to 10 carbon atoms, and
   Xⁱ represents a silylalkyl group which, when i=1, is represented by the following formula:
   in which R¹ is as defined above, R² represents an alkylene group having from 2 to 10 carbon atoms, R³ represents an alkyl group having from 1 to 10 carbon atoms, Xⁱ⁺¹ represents a hydrogen atom, an alkyl group having from 1 to 10 carbon atoms, an aryl group or the silylalkyl group defined above with i=i+1, i is an integer from 1 to 10 which represents the generation of said silylalkyl group and aⁱ is an integer from 0 to 3;
   where said radically polymerizable organic group contained in the component (A) is chosen from:
   organic groups containing a methacrylic group or an acrylic group and which are represented by the following formulae; and in which R⁴ represents a hydrogen atom or an alkyl group,
   R₅ represents an alkylene group having from 1 to 10 carbon atoms and organic groups containing a styryl group and which are represented by the following formula: in which R⁶ represents a hydrogen atom or an alkyl group, R⁷ represents an alkyl group having from 1 to 10 carbon atoms, R⁸ represents an alkylene group having from 1 to 10 carbon atoms, b is an integer from 0 to 4 and c has the value 0 or 1, such that, if c is 0,
   —(R⁸)_{c}— represents a bond.

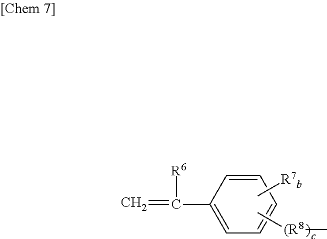

The monomer of vinyl type which is the component (A) in the vinyl polymer is a monomer of vinyl type which contains a radically polymerizable vinyl group.

There is no particular limitation as regards such a monomer.

The following are examples of this monomer of vinyl type: methyl methacrylate, ethyl methacrylate, n-propyl methacrylate, isopropyl methacrylate or a methacrylate of an analogous lower alkyl; glycidyl methacrylate; butyl methacrylate, butyl acrylate, n-butyl methacrylate, isobutyl methacrylate, tert-butyl acrylate, tert-butyl methacrylate, n-hexyl methacrylate, cyclohexyl methacrylate, 2-ethylhexyl acrylate, 2-ethylhexyl methacrylate, octyl methacrylate, lauryl methacrylate, stearyl acrylate, stearyl methacrylate or an analogous higher methacrylate; vinyl acetate, vinyl propionate or a vinyl ester of an analogous lower fatty acid; vinyl caproate, vinyl 2-ethylhexoate, vinyl laurate, vinyl stearate or an ester of an analogous higher fatty acid; styrene, vinyltoluene, benzyl methacrylate, phenoxyethyl methacrylate, vinylpyrrolidone or analogous vinylaromatic monomers; methacrylamide, N-methylolmethacrylamide, N-methoxymethylmethacrylamide, isobutoxymethoxymethacrylamide, N,N-dimethylmethacrylamide or analogous monomers of vinyl type which contain amide groups; hydroxyethyl methacrylate, hydroxypropyl methacrylate or analogous monomers of vinyl type which contain hydroxyl groups; acrylic acid, methacrylic acid, itaconic acid, crotonic acid, fumaric acid, maleic acid or analogous monomers of vinyl type which contain a carboxylic acid group; tetrahydrofurfuryl methacrylate, butoxyethyl methacrylate, ethoxydiethylene glycol methacrylate, polyethylene glycol methacrylate, polypropylene glycol monomethacrylate, hydroxybutyl vinyl ether, cetyl vinyl ether, 2-ethylhexyl vinyl ether or an analogous monomer of vinyl type with ether bonds; methacryloyloxypropyltrimethoxysilane, polydimethylsiloxane having a methacrylic group on one of its molecular ends, polydimethylsiloxane having a styryl group on one of its molecular ends, or an analogous silicone compound having unsaturated groups; butadiene; vinyl chloride; vinylidene chloride; methacrylonitrile; dibutyl fumarate; anhydrous maleic acid; anhydrous succinic acid; methacryl glycidyl ether; an organic salt of an amine, an ammonium salt, and an alkali metal salt of methacrylic acid, of itaconic acid, of crotonic acid, of maleic acid or of fumaric acid; a radically polymerizable unsaturated monomer having a sulfonic acid group, such as a styrenesulfonic acid group; a quaternary ammonium salt derived from methacrylic acid, such as 2-hydroxy-3-methacryloyloxypropyltrimethylammonium chloride; and a methacrylic acid ester of an alcohol having a tertiary amine group, such as a methacrylic acid ester of diethylamine. Multifunctional monomers of vinyl type can also be used.

The following represent examples of such compounds: trimethylolpropane trimethacrylate, pentaerythrityl trimethacrylate, ethylene glycol dimethacrylate, tetraethylene glycol dimethacrylate, polyethylene glycol dimethacrylate, 1,4-butanediol dimethacrylate, 1,6-hexanediol dimethacrylate, neopentyl glycol dimethacrylate, trimethylolpropane trioxyethyl methacrylate, tris(2-hydroxyethyl) isocyanurate dimethacrylate, tris(2-hydroxyethyl) isocyanurate trimethacrylate, polydimethylsiloxane capped with styryl groups having divinylbenzene groups on both ends, or analogous silicone compounds having unsaturated groups.

To facilitate the preparation of a mixture of the starting material for cosmetic products, the number-average molecular weight of the vinyl polymer which contains a carbosiloxane dendrimer can be chosen within the range between 3000 g/mol and 2 000 000 g/mol and preferably between 5000 g/mol and 800 000 g/mol. It can be a liquid, a gum, a paste, a solid, a powder or any other form. The preferred forms are solutions formed by dilution of a dispersion or of a powder in solvents, such as a silicone oil or an organic oil.

A vinyl polymer contained in the dispersion or the solution can have a concentration within a range of between 0.1% and 95% by weight and preferably between 5% and 70% by weight. However, to facilitate the handling and the preparation of the mixture, the range should preferably be between 10% and 60% by weight.

According to a preferred form, a vinyl polymer suitable for the invention can be one of the polymers described in the examples of the application EP 0 963 751.

According to a preferred embodiment, a vinyl polymer grafted with a carbosiloxane dendrimer can result from the polymerization:
of 0.1 to 99 parts by weight of one or more acrylate or methacrylate monomer(s); and
of 100 to 0.1 parts by weight of an acrylate or methacrylate monomer of a tris[tri(trimethylsiloxy)silylethyldimethylsiloxy]silylpropyl carbosiloxane dendrimer.

According to one embodiment, a vinyl polymer having at least one unit derived from carbosiloxane dendrimer can comprise a unit derived from tris[tri(trimethylsiloxy)silylethyldimethylsiloxy]silylpropyl corresponding to one of the formulae or carbosiloxane dendrimer

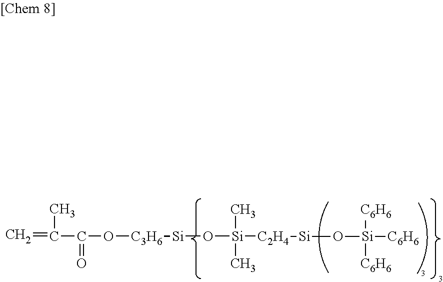

According to one embodiment, a vinyl polymer having at least one unit derived from carbosiloxane dendrimer can comprise a unit derived from tris[tri(trimethylsiloxy)silylethyldimethylsiloxy]silylpropyl carbosiloxane dendrimer corresponding to one of the formulae or

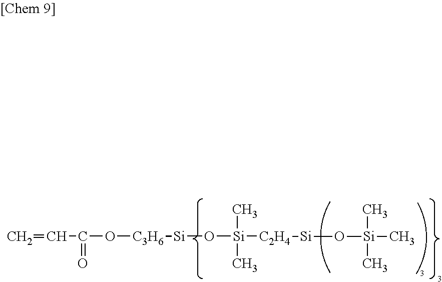

According to a preferred form, a vinyl polymer having at least one unit derived from carbosiloxane dendrimer used in the invention comprises at least one butyl acrylate monomer.

According to one embodiment, a vinyl polymer can additionally comprise at least one fluorinated organic group. A fluorinated vinyl polymer can be one of the polymers described in the examples of the application WO 03/045337.

According to a preferred embodiment, a grafted vinyl polymer within the meaning of the present invention can be conveyed in an oil or a mixture of oils, which is/are preferably volatile, chosen in particular from silicone oils and hydrocarbon oils and their mixtures.

According to a specific embodiment, a silicone oil suitable for the invention can be cyclopentasiloxane.

According to another specific embodiment, a hydrocarbon oil suitable for the invention can be isododecane.

Vinyl polymers grafted with at least one unit derived from carbosiloxane dendrimer which may be particularly suitable for the present invention are the polymers sold under the names TIB 4-100R, TIB 4-101®, TIB 4-120®, TIB 4-130®, TIB 4-200®, FA 4002 IDR (TIB 4-202R), TIB 4-220® and FA 4001 CM® (TIB 4-230®) by Dow Corning. Use will preferably be made of the polymers sold under the names FA 4002 IDR (TIB 4-202) and FA 4001 CM® (TIB 4-230®) by Dow Corning.

Preferably, the vinyl polymer grafted with at least one unit derived from carbosiloxane dendrimer which can be used in a composition of the invention is an acrylate/polytrimethylsiloxymethacrylate copolymer with the INCI name: Acrylates/Polytrimethyl Siloxymethacrylate Copolymer, in particular that sold in isododecane under the name Dow Corning FA 4002 IDR Silicone Acrylate by Dow Corning.

### IV. Silicone-Acrylate Copolymers

According to a specific embodiment, a composition used according to the invention can comprise, as hydrophobic film-forming polymer, at least one copolymer comprising carboxylate groups and polydimethylsiloxane groups.

The term “copolymer comprising carboxylate groups and polydimethylsiloxane groups” is understood to mean, in the present patent application, a copolymer obtained from (a) one or more carboxylic (acid or ester) monomers, and (b) one or more polydimethylsiloxane (PDMS) chains.

In the present patent application, the term “carboxylic monomer” means both carboxylic acid monomers and carboxylic acid ester monomers. Thus, the monomer (a) can be chosen, for example, from acrylic acid, methacrylic acid, maleic acid, fumaric acid, itaconic acid, crotonic acid, their esters and the mixtures of these monomers.

Mention may be made, as esters, of the following monomers: acrylate, methacrylate, maleate, fumarate, itaconate and/or crotonate. According to a preferred embodiment of the invention, the monomers in the form of esters are more particularly chosen from linear or branched, preferably C₁-C₂₄ and better still C₁-C₂₂, alkyl acrylates and methacrylates, the alkyl radical preferentially being chosen from methyl, ethyl, stearyl, butyl and 2-ethylhexyl radicals, and their mixtures.

Thus, according to a specific embodiment of the invention, the copolymer comprises, as carboxylate groups, at least one group chosen from acrylic acid, methacrylic acid, methyl, ethyl, stearyl, butyl or 2-ethylhexyl acrylate or methacrylate, and their mixtures.

In the present patent application, the term “polydimethylsiloxanes” (also known as organopolysiloxanes or, in abbreviation, PDMSs) is understood to denote, in accordance with what is generally accepted, any organosilicon polymer or oligomer having a linear structure, of variable molecular weight, obtained by polymerization and/or polycondensation of suitably functionalized silanes, and constituted essentially of a repetition of main units in which the silicon atoms are connected together by oxygen atoms (siloxane bond □Si—O—Si□), comprising methyl radicals directly bonded via a carbon atom to said silicon atoms. The PDMS chains which can be used in order to obtain the copolymer used according to the invention comprise at least one polymerizable radical group, preferably located on at least one of the ends of the chain, that is to say that the PDMS can have, for example, a polymerizable radical group on the two ends of the chain or have a polymerizable radical group on one end of the chain and a trimethylsilyl end group on the other end of the chain. The polymerizable radical group can in particular be an acrylic or methacrylic group, especially a CH₂═CR₁—CO—O—R² group, where R₁ represents a hydrogen or a methyl group and R₂ represents —CH₂—, —(CH₂)ₙ— with n═3, 5, 8 or 10, —CH₂—CH(CH₃)—CH₂—, —CH₂—CH₂—O—CH₂—CH₂—, —CH₂—CH₂—O—CH₂—CH₂—CH (CH₃)—CH₂—, —CH₂—CH₂—O—CH₂ or —CH₂—O—CH₂—CH₂—CH₂—.

The copolymers used in the composition of the invention are generally obtained according to the usual methods of polymerization and grafting, for example by radical polymerization (A) of a PDMS comprising at least one radically polymerizable group (for example on one of the ends of the chain or on both ends) and (B) of at least one carboxylic monomer, as described, for example, in the documents U.S. Pat. Nos. 5,061,481 and 5,219,560.

The copolymers obtained generally have a molecular weight ranging from approximately 3000 g/mol to 200 000 g/mol and preferably from approximately 5000 g/mol to 100 000 g/mol.

The copolymer used in the composition of the invention can be provided as is or in dispersed form in a solvent, such as lower alcohols comprising from 2 to 8 carbon atoms, for instance isopropyl alcohol, or oils, for instance volatile silicone oils (for example cyclopentasiloxane).

Mention may be made, as copolymers which can be used in the composition of the invention, for example, of copolymers of acrylic acid and of stearyl acrylate having polydimethylsiloxane grafts, copolymers of stearyl methacrylate having polydimethylsiloxane grafts, copolymers of acrylic acid and of stearyl methacrylate having polydimethylsiloxane grafts or copolymers of methyl methacrylate, of butyl methacrylate, of 2-ethylhexyl acrylate and of stearyl methacrylate having polydimethylsiloxane grafts. Mention may in particular be made, as copolymers which can be used in the composition of the invention, of the copolymers sold by Shin-Etsu under the names KP-561® (CTFA name: Acrylates/Dimethicone), KP-541®, where the copolymer is dispersed at 60% by weight in isopropyl alcohol (CTFA name: Acrylates/Dimethicone and Isopropyl Alcohol), and KP-545®, where the copolymer is dispersed at 30% in cyclopentasiloxane (CTFA name: Acrylates/Dimethicone and Cyclopentasiloxane).

According to a preferred embodiment of the invention, KP561® is preferably used; this copolymer is not dispersed in a solvent but is provided in waxy form, its melting point being approximately 30° C.

Mention may also be made of the polydimethylsiloxane-grafted acrylic acid copolymer dissolved in isododecane sold by Shin-Etsu under the name KP-550®.

According to a particularly preferred form, a composition according to the invention comprises, as hydrophobic film-forming polymer, at least one trimethylsiloxysilicate resin, such as those sold under the reference SR1000® by General Electric, under the reference TMS 803® by Wacker and under the names KF-7312@J by Shin-Etsu and DC 749® and DC 593® by Dow Corning.

Advantageously, a composition according to the invention comprises, as hydrophobic film-forming polymer, at least one trimethylsiloxysilicate resin, such as those sold under the reference SR1000® by General Electric, under the reference TMS 803® by Wacker and under the names KF-7312RJ by Shin-Etsu and DC749® and DC593® by Dow Corning.

### Linear Polyoxyalkylenated Polydimethylmethylsiloxane Emulsifying Surfactant

The composition in the form of a water-in-oil emulsion in accordance with the invention comprises at least one linear polyoxyalkylenated polydimethylmethylsiloxane with an HLB≤8, preferably linear polyoxypropylenated and/or polyoxyethylenated, in particular corresponding to the following formula (I):

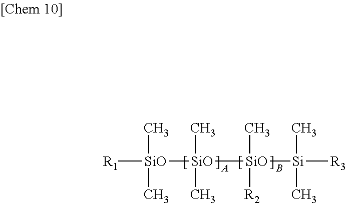

(I)
in which R¹, R² and R³, independently of one another, represent a C₁-C₆ alkyl radical or a —(CH₂)ₓ—(OCH₂CH₂)_{y}—(OCH₂CH₂CH₂)_{z}—OR₄ radical, at least one R₁, R₂ or R₃ radical not being an alkyl radical, R₄ being a hydrogen, a C₁-C₃ alkyl radical or a C₂-C₄ acyl radical;
A is an integer ranging from 0 to 200;
B is an integer ranging from 0 to 50; provided that A and B are not simultaneously equal to zero;
x is an integer ranging from 0 to 6;
y is an integer ranging from 1 to 30;
z is an integer ranging from 0 to 30.

Advantageously, a composition according to the invention comprises, as hydrophobic film-forming polymer, at least one trimethylsiloxysilicate resin, such as those sold under the reference SR1000® by General Electric, under the reference TMS 803® by Wacker and under the names KF-7312RJ by Shin-Etsu and DC749® and DC593® by Dow Corning.

### Linear Polyoxyalkylenated Polydimethylmethylsiloxane Emulsifying Surfactant

The composition in the form of a water-in-oil emulsion in accordance with the invention comprises at least one linear polyoxyalkylenated polydimethylmethylsiloxane with an HLB≤8, preferably linear polyoxypropylenated and/or polyoxyethylenated, in particular corresponding to the following formula (I):

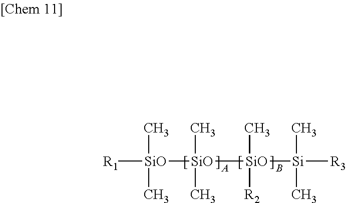

(I)
in which R¹, R² and R³, independently of one another, represent a C₁-C₆ alkyl radical or a —(CH₂)ₓ—(OCH₂CH₂)_{y}—(OCH₂CH₂CH₂)_{z}—OR₄ radical, at least one R₁, R₂ or R₃ radical not being an alkyl radical, R₄ being a hydrogen, a C₁-C₃ alkyl radical or a C₂-C₄ acyl radical;
A is an integer ranging from 0 to 200;
B is an integer ranging from 0 to 50; provided that A and B are not simultaneously equal to zero;
x is an integer ranging from 0 to 6;
y is an integer ranging from 1 to 30;
z is an integer ranging from 0 to 30.

According to a preferred embodiment of the invention, in the compound of formula (I), R₁=R₃=methyl radical, x=0 to 3 and R₄ is a hydrogen.

Mention may be made, as examples of compounds of formula (I), of the emulsifying surfactants with the following INCI names:
PEG/PPG-8/8 Dimethicone, such as the products sold under the trade names Silube J208-41®, Silube J208-61® and Silube J208-81® by;
PEG/PPG-18/18 Dimethicone used alone, such as the products sold under the trade names Andisil SP 1818® by AB Specialty Silicones, Jeesilc DMC 19® by Jeen International Corporation, Silsurf J-1013-V-CG® by Siltech LLC or Xiameter OFX-0190 Fluid® by Dow Chemical Company;
the Cyclopentasiloxane and PEG/PPG-18/18 Dimethicone mixture, such as the products sold under the trade names Dowsil 5225C Formulation Aid® by Dow Chemical; Emusil WO-5115® by Innospec Performance Chemicals; Gransurf 10C® by Grant Industries Inc.; Jeesilc DMC522® by Jeen International Corporation; Silsurf 400R® by Siltech LLC;
the Cyclotetrasiloxane and Cyclopentasiloxane and PEG/PPG-18/18 Dimethicone mixture, such as the commercial products sold under the names Dowsil 3225C Formulation Aid® by Dow Chemical; Emulsil WO-3115® by Innospec Performance Chemicals; Jeesilc DMC252® and Jeesilc DMC322® by Jeen International Corporation;
the Dimethicone and PEG/PPG-18/18 Dimethicone mixture, such as the commercial products Dowsil ES-5226 DM Formulation Aid® and Dowsil ES-5227 DM Formulation Aid® from Dow Chemical; Gransurf 50C® and Gransurf 50C-HM® from Grant Industries Inc.; and X-22-6711D from Shin-Etsu;
the PEG/PPG-19/19 Dimethicone and C₁₃-C₁₆ Isoparaffin and C₁₀-C₁₃ Isoparaffin mixture, such as the commercial product Dow Corning® BY 25-337 from Dow Chemical;
PEG-3 Dimethicone, such as the commercial product KF-6015® from Shin-Etsu Chemical Co. Ltd.;
PEG-10 Dimethicone, such as the commercial products KF-6017® from Shin-Etsu Chemical Co. Ltd.; Serasol SC 86® and Serasol SC 86A® from KCC Corporation;
their mixtures.

According to a specific form of the invention, the composition of the invention comprises at least PEG/PPG-18/18 Dimethicone, preferably in combination with a silicone oil, in particular a linear silicone oil of the dimethicone type (INCI name: Dimethicone and PEG/PPG-18/18 Dimethicone) such as the commercial products

Dowsil ES-5226 DM Formulation Aid® and Dowsil ES-5227 DM Formulation Aid® from Dow Chemical; Gransurf 50C® and Gransurf 50C-HM® from Grant Industries Inc.; and X-22-6711D from Shin-Etsu.

According to another specific form, the composition of the invention comprises at least one mixture of PEG/PPG-18/18 Dimethicone and PEG-10 Dimethicone, preferably a mixture of PEG/PPG-18/18 Dimethicone, dimethicone and PEG-10 Dimethicone.

The linear polyoxyalkylenated polydimethylmethylsiloxane(s) in accordance with the invention are preferably present in the composition of the invention at concentrations ranging from 0.1% to 10% by weight, more preferentially ranging from 0.5% to 7% by weight and more preferentially still ranging from 1% to 4% by weight, with respect to the total weight of the composition.

### Vitamin B3, Derivatives and Salts

The term “vitamin B3” is understood to mean any molecule having as general formula:
in which R is —CONH₂ (i.e. niacinamide, isoniacinamide), —COOH (i.e.: nicotinic acid) or —CH₂ OH (i.e.: nicotinyl alcohol), and also its derivatives and organic or inorganic acid salts or its inorganic or organic base salts, such as those mentioned above. Mention may be made, as examples of derivatives of vitamin B3, of:
   esters of nicotinic acid, such as those with the following INCI names: Niacinamide Ascorbate, Niacinamide Glycolate, Niacinamide Hydroxybenzoate, Niacinamide Hydroxycitrate, Niacinamide Lactate, Niacinamide Malate, Niacinamide Mandalate, Niacinamide Salicylate, Niacinamide Thioctate, supplied by Bioderma Tech. Co. Ltd.;
   quaternary ammonium salts, such as Methyl Niacinamide Chloride (INCI name), such as the commercial product MNA Chloride® from Pharmena;
   the reaction products of niacinamide with polypeptides, such as those resulting from yeasts: INCI name Niacinamide/Yeast Polypeptide, such as the commercial product sold under the commercial name Vitazyme B₃® by Arch Personal Care Products, L. P./Lonza Personal Care.

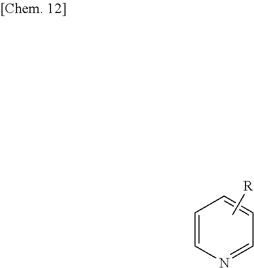

Use will more particularly be made of niacinamide, like the commercial products sold under the name:
Niacinamide PC® (DSM Nutritional Products Inc.)
OriStar NA® (Orient Stars LLC)
RonaCare Nicotinamide® (Merck KGaA/EMD Chemicals)
RonaCare Nicotinamide® (EMD Chemicals)

Vitamin B3 and/or one of its derivatives are preferably present in the compositions according to the invention in concentrations as active material ranging from 0.01% to 20% by weight, better still from 0.1% to 10% by weight, more preferably still from 0.5% to 5% by weight, with respect to the total weight of the composition.

### Pigments

According to a specific form of the invention, the composition additionally comprises at least one pigment.

The term “pigments” is understood to mean white or colored and inorganic or organic particles which are insoluble in an aqueous medium and which are intended to color and/or opacify the resulting composition and/or deposit. These pigments can be white or colored and inorganic and/or organic.

Preferably, the composition comprises at least 5% by weight of pigment(s), more preferentially from 5% to 40% by weight of pigment(s), in particular from 10% to 30% by weight of pigment(s) and preferably from 9% to 20% by weight of pigment(s), with respect to the total weight of said composition.

According to a specific embodiment, the pigments used according to the invention are chosen from inorganic pigments.

The term “inorganic pigment” is understood to mean any pigment which satisfies the definition of Ullmann's Encyclopedia in the chapter “Pigments, Inorganic”. Mention may be made, among the inorganic pigments of use in the present invention, of zirconium oxide or cerium oxide, and also zinc oxide, iron oxide (black, yellow or red) or chromium oxide, manganese violet, ultramarine blue, chromium hydrate and ferric blue, titanium dioxide, or metal powders, such as aluminum powder and copper powder. The following inorganic pigments can also be used: Ta₂O₅, Ti₃O₅, Ti₂O₃, TiO, ZrO₂ as a mixture with TiO₂, ZrO₂, Nb2O5, CeO₂ and ZnS.

The size of the pigment of use in the context of the present invention is generally greater than 100 nm and can range up to 10 μm, preferably from 200 nm to 5 μm and more preferentially from 300 nm to 1 μm.

According to a specific form of the invention, the pigments exhibit a size characterized by a D [50] of greater than 100 nm and which can range up to 10 μm, preferably from 200 nm to 5 μm and more preferentially from 300 nm to 1 μm.

The sizes are measured by static light scattering using a commercial particle size analyser of MasterSizer 3000® type from Malvern, making it possible to determine the particle size distribution of all of the particles over a wide range which can extend from 0.01 μm to 1000 μm. The data are processed on the basis of the conventional Mie scattering theory. This theory is the most suitable for size distributions ranging from the submicronic to multimicronic; it makes it possible to determine an effective particle diameter. This theory is described in particular in the publication by Van de Hulst, H. C., Light Scattering by Small Particles, Chapters 9 and 10, Wiley, New York, 1957.

D[50] represents the maximum size presented by 50% by volume of the particles.

In the context of the present invention, the inorganic pigments are more particularly iron oxide and/or titanium dioxide. Mention may more particularly be made, by way of examples, of titanium dioxides and iron oxides, which are coated with aluminum stearoyl glutamate, for example sold under the reference NAIR by Miyoshi Kasei.

Mention may also be made, as inorganic pigments which can be used in the invention, of pearlescent agents.

The term “pearlescent agents” should be understood as meaning colored particles of any shape, which are or are not iridescent, in particular produced by certain mollusks in their shells or else synthesized, and which 4

The pearlescent agents can be chosen from pearlescent pigments, such as titanium oxide-coated mica covered with an iron oxide, titanium oxide-coated mica covered with bismuth oxychloride, titanium oxide-coated mica covered with chromium oxide, titanium oxide-coated mica covered with an organic dye and also pearlescent pigments based on bismuth oxychloride. They can also be mica particles, at the surface of which are superimposed at least two successive layers of metal oxides and/or of organic colorants.

Mention may also be made, as examples of pearlescent agents, of natural mica covered with titanium oxide, with iron oxide, with natural pigment or with bismuth oxychloride.

Mention may be made, among the commercially available pearlescent agents, of the Timica®, Flamenco® and Duochrome® pearlescent agents (on a mica base) sold by Engelhard, the Timiron® pearlescent agents sold by Merck, the Prestige® pearlescent agents on a mica base sold by Eckart and the Sunshine® pearlescent agents on a synthetic mica base sold by Sun Chemical.

The pearlescent agents can more particularly have a yellow, pink, red, bronze, orangey, brown, gold and/or coppery color or glint.

Mention may in particular be made, by way of illustration of the pearlescent agents which can be employed in the context of the present invention, of gold-colored pearlescent agents sold in particular by Engelhard under the names Brilliant Gold 212G® (Timica), Gold 222C® (Cloisonne), Sparkle Gold® (Timica), Gold 4504® (Chromalite) and Monarch Gold 233X® (Cloisonne); bronze pearlescent agents sold in particular by Merck under the names Bronze Fine® (17384) (Colorona) and Bronze® (17353) (Colorona) and by Engelhard under the name Super Bronze (Cloisonne); orange pearlescent agents sold in particular by Engelhard under the names Orange 363C® (Cloisonne) and Orange MCR 101® (Cosmica) and by Merck under the names Passion Orange® (Colorona) and Matte Orange (17449)® (Microna); brown-colored pearlescent agents sold in particular by Engelhard under the names Nu-Antique Copper 340XBR (Cloisonne) and Brown CL4509® (Chromalite); pearlescent agents with a copper glint sold in particular by Engelhard under the name Copper 340AR (Timica); pearlescent agents with a red glint sold in particular by Merck under the name Sienna Fine® (17386) (Colorona); pearlescent agents with a yellow glint sold in particular by Engelhard under the name Yellow (4502)® (Chromalite); red-colored pearlescent agents with a gold glint sold in particular by Engelhard under the name Sunstone G012® (Gemtone); pink pearlescent agents sold in particular by Engelhard under the name Tan Opale G005® (Gemtone); black pearlescent agents with a gold glint sold in particular by Engelhard under the name Nu Antique Bronze 240 ABR (Timica); blue pearlescent agents sold in particular by Merck under the name Matte Blue® (17433) (Microna); white pearlescent agents with a silvery glint sold in particular by Merck under the name Xirona Silver® and golden green pinkish orangey pearlescent agents sold in particular by Merck under the name Indian Summer® (Xirona); and their mixtures.

Mention may also be made, among the pigments which can be used according to the invention, of those having an optical effect different from a simple conventional coloring effect, that is to say a unified and stabilized effect such as is produced by conventional colorants, such as, for example, monochromatic pigments. Within the meaning of the invention, the term “stabilized” means devoid of effect of variability of the color with the angle of observation or also in response to a temperature change.

For example, this material can be chosen from particles with a metallic glint, goniochromatic coloring agents, diffractive pigments, thermochromic agents, optical brighteners, and also fibers, in particular interference fibers. Of course, these various materials can be combined so as to provide the simultaneous display of two effects, indeed even of a novel effect in accordance with the invention.

The particles with a metallic glint which can be used in the invention are in particular chosen from:
particles of at least one metal and/or of at least one metal derivative,
particles comprising a monomaterial or multimaterial organic or inorganic substrate, at least partially covered with at least one layer with a metallic glint comprising at least one metal and/or at least one metal derivative, and
the mixtures of said particles.

Mention may be made, among the metals which can be present in said particles, for example, of Ag, Au, Cu, Al, Ni, Sn, Mg, Cr, Mo, Ti, Zr, Pt, Va, Rb, W, Zn, Ge, Te, Se and their mixtures or alloys. Ag, Au, Cu, Al, Zn, Ni, Mo, Cr and their mixtures or alloys (for example, bronzes and brasses) are preferred metals.

The term “metal derivatives” denotes compounds derived from metals, in particular oxides, fluorides, chlorides and sulfides.

Mention may be made, by way of illustration of these particles, of aluminum particles, such as those sold under the names Starbrite 1200 EAC® by Silberline and Metalure® by Eckart.

Mention may also be made of metal powders formed of copper or alloy mixtures, such as the references 2844 sold by Radium Bronze, metal pigments, such as aluminum or bronze, for example those sold under the names Rotosafe 700® from Eckart, silica-coated aluminum particles sold under the name Visionaire Bright Silver® from Eckart and particles formed of metal alloy, such as powders formed of bronze (copper and zinc alloy) coated with silica sold under the name Visionaire Bright Natural Gold® from Eckart.

They may also be particles comprising a glass substrate, such as those sold by Nippon Sheet Glass under the names Microglass Metashine®.

The goniochromatic coloring agent can be chosen, for example, from multilayer interference structures and liquid crystal coloring agents.

Examples of symmetrical interference multilayer structures which can be used in compositions produced in accordance with the invention are, for example, the following structures: Al/SiO₂/Al/SiO₂/Al, pigments having this structure being sold by DuPont De Nemours; Cr/MgF₂/Al/MgF₂/Cr, pigments having this structure being sold under the name Chromaflair® by Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl® by BASF; MOS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by Merck (Darmstadt). By way of example, these pigments can be pigments with a silica/titanium oxide/tin oxide structure sold under the name Xirona Magic® by Merck, pigments with a silica/brown iron oxide structure sold under the name Xirona Indian Summer® by Merck and pigments with a silica/titanium oxide/mica/tin oxide structure sold under the name Xirona Caribbean Blue® by Merck. Mention may also be made of the Infinite Colors pigments from Shiseido. Different effects are obtained according to the thickness and the nature of the various layers. Thus, with the structure Fe₂O₃/SiO₂/Al/SiO₂/Fe₂O₃, the color changes from green-golden to red-grey for SiO₂ layers of 320 to 350 nm; from red to golden for SiO₂ layers of 380 to 400 nm; from purple to green for SiO₂ layers of 410 to 420 nm; and from copper to red for SiO₂ layers of 430 to 440 nm.

Mention may be made, as examples of pigments with a polymeric multilayer structure, of those sold by 3M under the name Color Glitter®.

Use may be made, as liquid crystal goniochromatic particles, for example, of those sold by Chenix and of those sold under the name Helicone® HC by Wacker.

### Hydrophobic Coated Pigments

According to a specific form of the invention, the compositions according to the invention comprise at least one pigment coated with at least one lipophilic or hydrophobic compound and in particular as described in detail below.

This type of pigment is particularly advantageous insofar as it may be considered in a large amount together with a large amount of water. What is more, insofar as they are treated with a hydrophobic compound, they show a predominant affinity for the oily gelled phase, which can then convey them.

Of course, the compositions according to the invention can in parallel contain uncoated pigments.

The coating can also comprise at least one additional non-lipophilic compound.

Within the meaning of the invention, the “coating” of a pigment according to the invention generally denotes the total or partial surface treatment of the pigment with a surface agent, absorbed on, adsorbed on or grafted to said pigment.

The surface-treated pigments can be prepared according to surface treatment techniques of chemical, electronic, mechanochemical or mechanical nature which are well known to a person skilled in the art. Commercial products can also be used.

The surface agent can be absorbed on, adsorbed on or grafted to the pigments by solvent evaporation, chemical reaction and creation of a covalent bond.

According to one alternative form, the surface treatment consists of a coating of the pigments.

The coating can represent from 0.1% to 20% by weight and in particular from 0.5% to 5% by weight of the total weight of the coated pigment.

The coating can be produced, for example, by adsorption of a liquid surface agent at the surface of the solid particles by simple mixing with stirring of the particles and of said surface agent, optionally with heating, prior to the incorporation of the particles in the other ingredients of the makeup or care composition.

The coating can be produced, for example, by chemical reaction of a surface agent with the surface of the solid pigment particles and creation of a covalent bond between the surface agent and the particles. This method is described in particular in the patent U.S. Pat. No. 4,578,266.

The chemical surface treatment can consist in diluting the surface agent in a volatile solvent, in dispersing the pigments in this mixture and in then slowly evaporating the volatile solvent, so that the surface agent is deposited at the surface of the pigments.

### Lipophilic or Hydrophobic Treatment Agent

When the pigment comprises a lipophilic or hydrophobic coating, the latter is preferably present in the fatty phase of the composition according to the invention.

According to a specific embodiment of the invention, the pigments can be coated according to the invention with at least one compound chosen from silicone surface agents; fluorinated surface agents; fluorosilicone surface agents; metal soaps; N-acylamino acids or their salts; lecithin and its derivatives; isopropyl titanium triisostearate; isostearyl sebacate; natural vegetable or animal waxes; polar synthetic waxes; fatty esters; phospholipids; and their mixtures.

### Silicone Surface Agent

According to a specific embodiment, the pigments can be completely or partially surface-treated with a compound of silicone nature.

The silicone surface agents can be chosen from organopolysiloxanes, silane derivatives, silicone-acrylate copolymers, silicone resins and their mixtures.

The term “organopolysiloxane compound” is understood to mean a compound having a structure comprising an alternation of silicon atoms and oxygen atoms and comprising organic radicals bonded to the silicon atoms.

### Nonelastomeric Organopolysiloxane

Mention may in particular be made, as nonelastomeric organopolysiloxanes, of polydimethylsiloxanes, polymethylhydrosiloxanes and polyalkoxydimethylsiloxanes.

The alkoxy group can be represented by the R—O— radical such that R represents methyl, ethyl, propyl, butyl or octyl, 2-phenylethyl, 2-phenylpropyl or 3,3,3-trifluoropropyl radicals, aryl radicals, such as phenyl, tolyl or xylyl, or substituted aryl radicals, such as phenylethyl.

One method which makes it possible to surface-treat pigments with a polymethylhydrosiloxane consists in dispersing the pigments in an organic solvent and in then adding the silicone compound. On heating the mixture, covalent bonds are created between the silicone compound and the surface of the pigment.

According to a preferred embodiment, the silicone surface agent can be a nonelastomeric organopolysiloxane, in particular chosen from polydimethylsiloxanes.

### Alkylsilanes and Alkoxysilanes

Silanes having alkoxy functionality are described in particular by Witucki in A Silane Primer, Chemistry and Applications of Alkoxysilanes, Journal of Coatings Technology, 65, 822, pages 57-60, 1993.

Alkoxysilanes, such as the alkyltriethoxysilanes and the alkyltrimethoxysilanes sold under the references Milquet A-137® (OSI Specialities) and Prosil 9202® (PCR), can be used for coating the pigments.

The use of alkylpolysiloxanes having a reactive end group, such as alkoxy, hydroxyl, halogen, amino or imino, is described in the application JP H07-196946. They are also suitable for treating the pigments.

### Silicone-Acrylate Polymers

Use may be made of grafted silicone-acrylic polymers having a silicone backbone as described in the patents U.S. Pat. Nos. 5,725,882, 5,209,924, 4,972,037, 4,981,903, 4,981,902, 5,468,477 and in the patents U.S. Pat. No. 5,219,560 and EP 0 388 582.

Other silicone-acrylate polymers can be silicone polymers comprising, in their structure, the unit of following formula (II): (II)
in which the G₁ radicals, which are identical or different, represent hydrogen or a C₁-C₁₀ alkyl radical or also a phenyl radical; the G₂ radicals, which are identical or different, represent a C₁-C₁₀ alkylene group; G₃ represents a polymeric residue resulting from the (homo) polymerization of at least one ethylenically unsaturated anionic monomer; G₄ represents a polymeric residue resulting from the (homo) polymerization of at least one ethylenically unsaturated hydrophobic monomer; m and n are equal to 0 or 1; a is an integer ranging from 0 to 50; b is an integer which can be of between 10 and 350 and c is an integer ranging from 0 to 50, with the proviso that one of the parameters a and c is other than 0.

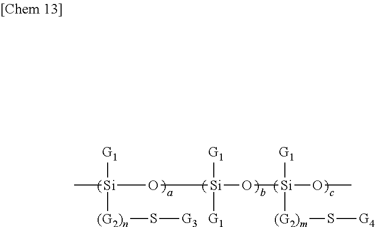

Other silicone-acrylate polymers can be silicone polymers comprising, in their structure, the unit of following formula (II): (II)
in which the G₁ radicals, which are identical or different, represent hydrogen or a C₁-C₁₀ alkyl radical or also a phenyl radical; the G₂ radicals, which are identical or different, represent a C₁-C₁₀ alkylene group; G₃ represents a polymeric residue resulting from the (homo) polymerization of at least one ethylenically unsaturated anionic monomer; G₄ represents a polymeric residue resulting from the (homo) polymerization of at least one ethylenically unsaturated hydrophobic monomer; m and n are equal to 0 or 1; a is an integer ranging from 0 to 50; b is an integer which can be of between 10 and 350 and c is an integer ranging from 0 to 50, with the proviso that one of the parameters a and c is other than 0.

Preferably, the unit of formula (I) above exhibits at least one and more preferentially still all of the following characteristics:
the G₁ radicals denote an alkyl radical, preferably the methyl radical;
n is nonzero, and the G₂ radicals represent a divalent C₁-C₃ radical, preferably a propylene radical;
G₃ represents a polymeric radical resulting from the (homo) polymerization of at least one monomer of the ethylenically unsaturated carboxylic acid type, preferably acrylic acid and/or methacrylic acid;
G₄ represents a polymeric radical resulting from the (homo) polymerization of at least one monomer of the (C₁-C₁₀)alkyl(meth)acrylate type, preferably of the isobutyl or methyl (meth)acrylate type.

Examples of silicone polymers corresponding to the formula (I) are in particular polydimethylsiloxanes (PDMSs) to which mixed polymer units of the poly(meth)acrylic acid type and of the polymethyl (meth)acrylate type are grafted via a thiopropylene-type connecting link.

Other examples of silicone polymers corresponding to the formula (I) are in particular polydimethylsiloxanes (PDMSs) to which polymer units of the polyisobutyl (meth)acrylate type are grafted via a thiopropylene-type connecting link.

### Silicone Resins

The silicone surface agent may be chosen from silicone resins, such as those defined above.

### Fluorinated Surface Agent

The pigments can be completely or partially surface-treated with a compound of fluorinated nature.

The fluorinated surface agents can be chosen from perfluoroalkyl phosphates, perfluoropolyethers, polytetrafluoroethylenes (PTFEs), perfluoroalkanes, perfluoroalkyl silazanes, polyhexafluoropropylene oxides or polyorganosiloxanes comprising perfluoroalkyl perfluoropolyether groups.

The term “perfluoroalkyl radical” is understood to mean an alkyl radical in which all the hydrogen atoms have been replaced with fluorine atoms.

Perfluoropolyethers are described in particular in the patent application EP 0 486 135 and are sold under the trade name Fomblin by Montefluos.

Perfluoroalkyl phosphates are described in particular in the application JP H05-86984. The perfluoroalkyl phosphate diethanolamines sold by Asahi Glass under the reference Asahi Guard AG530® can be used.

Mention may be made, among the linear perfluoroalkanes, of perfluorocycloalkanes, perfluoro (alkylcycloalkanes), perfluoropolycycloalkanes, perfluorinated aromatic hydrocarbons (perfluoroarenes) and organoperfluorinated hydrocarbon compounds comprising at least one heteroatom.

Mention may be made, among the perfluoroalkanes, of the series of the linear alkanes, such as perfluorooctane, perfluorononane or perfluorodecane.

Mention may be made, among the perfluorocycloalkanes and perfluoro (alkylcycloalkanes), of perfluorodecalin, sold under the name Flutec PP5 GMP by Rhodia, perfluoro (methyldecalin) or perfluoro ((C₃-C₅)alkylcyclohexanes), such as perfluoro (butylcyclohexane).

Mention may be made, among the perfluoropolycycloalkanes, of bicyclo[3.3.1]nonane derivatives, such as perfluorotrimethylbicyclo[3.3.1]nonane, adamantane derivatives, such as perfluorodimethyladamantane, and perfluorinated derivatives of hydrogenated as phenanthrene, such tetracosafluorotetradecahydrophenanthrene.

Mention may be made, among the perfluoroarenes, of perfluorinated derivatives of naphthalene, such as perfluoronaphthalene and perfluoro-1-methylnaphthalene.

Mention may be made, as examples of commercial references of pigments treated with a fluorinated compound, of:
yellow iron oxide/perfluoroalkyl phosphate, sold under the reference PF 5 Yellow 601® by Daito Kasei;
red iron oxide/perfluoroalkyl phosphate, sold under the reference PF 5 Red R 516L® by Daito Kasei;
black iron oxide/perfluoroalkyl phosphate, sold under the reference PF 5 Black BL 100® by Daito Kasei;
titanium dioxide/perfluoroalkyl phosphate, sold under the reference PF 5 TiO₂ CR 50® by Daito Kasei;
yellow iron oxide/perfluoropolymethyl isopropyl ether, sold under the reference Iron Oxide Yellow BF-25-3® by Toshiki;
DC Red 7/perfluoropolymethyl isopropyl ether, sold under the reference D&C Red 7 FHC® by Cardre Inc.; and
DC Red 6/PTFE, sold under the reference T 9506® by Warner-Jenkinson.

### Fluorosilicone Surface Agent

The pigments can be completely or partially surface-treated with a compound of fluorosilicone nature.

The fluorosilicone compound can be chosen from perfluoroalkyl dimethicones, perfluoroalkylsilanes and perfluoroalkyltrialkoxysilanes.

Mention may be made, as perfluoroalkylsilanes, of the products
LP-IT® and LP-4®, sold by Shin-Etsu Silicone. The perfluoroalkyl dimethicones can be represented by the following formula:

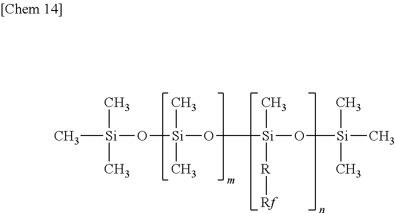

in which:
R represents a linear or branched divalent alkyl group having from 1 to 6 carbon atoms, preferably a divalent methyl, ethyl, propyl or butyl group;
Rf represents a perfluoroalkyl radical having from 1 to 9 carbon atoms and preferably from 1 to 4 carbon atoms;
m is chosen between 0 and 150 and preferably between 20 and 100; and
n is chosen between 1 and 300 and preferably between 1 and 100.

The fluorosilicone compound can be chosen from perfluoroalkyl dimethicones, perfluoroalkylsilanes and perfluoroalkyltrialkoxysilanes.

Mention may be made, as perfluoroalkylsilanes, of the products
LP-IT® and LP-4T®, sold by Shin-Etsu Silicone. The perfluoroalkyl dimethicones can be represented by the following formula:

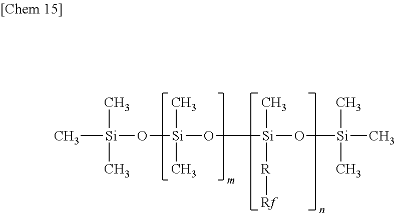

in which:
R represents a linear or branched divalent alkyl group having from 1 to 6 carbon atoms, preferably a divalent methyl, ethyl, propyl or butyl group;
Rf represents a perfluoroalkyl radical having from 1 to 9 carbon atoms and preferably from 1 to 4 carbon atoms;
m is chosen between 0 and 150 and preferably between 20 and 100; and
n is chosen between 1 and 300 and preferably between 1 and 100.

Mention may be made, as examples of commercial references of pigments treated with a fluorosilicone compound, of titanium dioxide/fluorosilicone, sold under the reference Fluorosil Titanium Dioxide 100TAR by Advanced Dermaceuticals International Inc.

### Other Lipophilic Surface Agents

The hydrophobic treatment agent can also be chosen from:
(i) metal soaps, such as aluminum dimyristate and the aluminum salt of hydrogenated tallow glutamate.

Mention may in particular be made, as metal soaps, of metal soaps of fatty acids having from 12 to 22 carbon atoms and in particular those having from 12 to 18 carbon atoms.

The metal of the metal soap can in particular be zinc or magnesium.

Use may be made, as metal soap, of zinc laurate, magnesium stearate, magnesium myristate, zinc stearate and their mixtures.

The hydrophobic treatment agent can also be chosen from ii) fatty acids, such as lauric acid, myristic acid, stearic acid or palmitic acid.

The hydrophobic treatment agent can also be chosen from iii)N-acylated amino acids or their salts, which can comprise an acyl group having from 8 to 22 carbon atoms, such as, for example, a 2-ethylhexanoyl, caproyl, lauroyl, myristoyl, palmitoyl, stearoyl or cocoyl group.

The amino acid can, for example, be lysine, glutamic acid or alanine.

The salts of these compounds can be the aluminum, magnesium, calcium, zirconium, zinc, sodium or potassium salts.

Thus, according to a particularly preferred embodiment, an N-acylated amino acid derivative can in particular be a glutamic acid derivative and/or one of its salts and more particularly a stearoyl glutamate, such as, for example, aluminum stearoyl glutamate.

The hydrophobic treatment agent can also be chosen from iv) lecithin and its derivatives.

The hydrophobic treatment agent can also be v) isopropyl titanium triisostearate.

Mention may be made, as examples of pigments treated with isopropyl titanium triisostearate (ITT), of those sold under the commercial references BWBO—I2® (iron oxide CI77499 and isopropyl titanium triisostearate), BWYO—I2® (iron oxide CI77492 and isopropyl titanium triisostearate) and BWRO—I2® (iron oxide CI77491 and isopropyl titanium triisostearate) by Kobo.

The hydrophobic treatment agent can also be vi) isostearyl sebacate.

The hydrophobic treatment agent can also be chosen from vii) natural vegetable or animal waxes or polar synthetic waxes.

The hydrophobic treatment agent can also be chosen from viii) fatty esters, in particular jojoba esters.

The hydrophobic treatment agent can also be chosen from ix)phospholipids.

The waxes mentioned in the compounds cited above can be those generally used in the cosmetics field, as are defined subsequently.

They can in particular be hydrocarbon, silicone and/or fluorinated, optionally comprising ester or hydroxyl functional groups. They can also be of natural or synthetic origin.

The term “polar wax” is understood to mean a wax containing chemical compounds comprising at least one polar group. Polar groups are well known to a person skilled in the art; they can, for example, be alcohol, ester or carboxylic acid groups. Polyethylene waxes, paraffin waxes, microcrystalline waxes, ozokerite or Fischer-Tropsch waxes are not included among polar waxes.

In particular, polar waxes have a mean Hansen solubility parameter δa at 25° C. such that δa>0 (J/cm³)^{1/2} and better still δa>1 (J/cm³)^{1/2}:

$${δ}_{a} = \sqrt{{δ}_{p}^{2} + {δ}_{h}^{2}}$$

where δp and δh are, respectively, the polar contributions and the contributions of types of specific interactions to the Hansen solubility parameters.

In particular, polar waxes have a mean Hansen solubility parameter δa at 25° C. such that δa>0 (J/cm³)^{1/2} and better still δa>1 (J/cm³)^{1/2}:

$${δ}_{a} = \sqrt{{δ}_{p}^{2} + {δ}_{h}^{2}}$$

where δp and δh are, respectively, the polar contributions and the contributions of types of specific interactions to the Hansen solubility parameters.

The definition of solvents in the three-dimensional solubility space according to Hansen is described in the paper by C. M. Hansen, The three-dimensional solubility parameters, J. Paint Technol., 39, 105 (1967):
δh characterizes the forces of specific interactions (such as hydrogen, acid/base or donor/acceptor bonds, and the like);
δp characterizes the Debye interaction forces between permanent dipoles and also the Keesom interaction forces between induced dipoles and permanent dipoles.

The parameters δp and δh are expressed in (J/cm³)^{1/2}.

A polar wax is formed in particular of molecules comprising, besides carbon and hydrogen atoms in their chemical structure, heteroatoms (such as O, N and P).

Mention may in particular be made, as nonlimiting illustration of these polar waxes, of natural polar waxes, such as beeswax, lanolin wax, orange wax, lemon wax and Chinese insect waxes, rice bran wax, carnauba wax, candelilla wax, ouricury wax, cork fiber wax, sugar cane wax, Japan wax, sumac wax or montan wax.

According to a specific embodiment, the pigments can be coated with at least one compound chosen from silicone surface agents; fluorinated surface agents; N-acylated amino acids or their salts; isopropyl titanium triisostearate; natural vegetable or animal waxes; fatty esters; and their mixtures.

According to a particularly preferred embodiment, the pigments can be coated with an N-acylated amino acid and/or one of its salts, in particular with a glutamic acid derivative and/or one of its salts, or with a fatty ester, in particular with a jojoba ester.

According to a more particularly preferred embodiment, the pigments can be coated with an N-acylated amino acid and/or one of its salts, in particular with a glutamic acid derivative and/or one of its salts, especially a stearoyl glutamate, such as, for example, aluminum stearoyl glutamate.

Mention may more particularly be made, as examples of coated pigments according to the invention, of titanium dioxides and iron oxides coated with aluminum stearoyl glutamate, for example sold under the reference NAI by Miyoshi Kasei.

Pigments not Coated with a Hydrophobic Compound

As stated above, a composition can additionally contain pigments not coated with a lipophilic or hydrophobic compound.

These other pigments can be coated with a hydrophilic compound or be uncoated.

These pigments can be inorganic pigments, in particular as defined above.

These pigments can also be organic pigments.

The term “organic pigment” is understood to mean any pigment which satisfies the definition of Ullmann's Encyclopedia in the chapter “Pigments, Organic”. The organic pigment can in particular be chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane or quinophthalone compounds.

The organic pigment(s) can be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as are described in the patent FR 2 679 771.

These pigments can also be in the form of composite pigments, such as are described in the patent EP 1 184 426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed on insoluble particles, the assembly thus obtained remaining insoluble during use.

The inorganic substrates on which the dyes are adsorbed are, for example, alumina, silica, calcium sodium borosilicate or calcium aluminum borosilicate, and aluminum.

Mention may be made, among the organic dyes, of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053) or D&C Blue 1 (CI 42 090).

Mention may be made, as examples of lakes, of the product known under the name D&C Red 7 (CI 15 850:1).

### Nature of the Hydrophilic Coating

As stated above, these other pigments can be coated with a hydrophilic compound.

Said hydrophilic compound making it possible to surface treat a pigment in order to optimize its dispersion in the gelled aqueous phase is more particularly chosen from biological polymers, carbohydrates, polysaccharides, polyacrylates or polyethylene glycol derivatives.

Mention may be made, as examples of biological polymers, of polymers based on monomers of carbohydrate type.

Mention may more particularly be made of biosaccharide gum; chitosans and their derivatives, such as butoxy chitosan, carboxymethyl chitosan, carboxybutyl chitosan, chitosan gluconate, chitosan adipate, chitosan glycolate, chitosan lactate, and the like; chitins and their derivatives, such as carboxymethyl chitin or chitin glycolate; cellulose and its derivatives, such as cellulose acetate; microcrystalline cellulose; distarch phosphate; sodium hyaluronate; soluble proteoglycans; galactoarabinans; glycosaminoglycans; glycogen; sclerotium gum; dextran; starch and its derivatives; and their mixtures.

Mention may in particular be made, as examples of carbohydrates, of polyhydroxyaldehydes or polyhydroxyketones, of general formula: Cₓ(H₂O)_{y} in which x and y can range from 1 to 1 000 000.

The carbohydrates can be monosaccharides, disaccharides or polysaccharides.

Mention may in particular be made, as examples of carbohydrates, of amylodextrins, beta-glucans, cyclodextrins, modified corn starch, glycogen, hyaluronic acid, hydroxypropylcyclodextrin, lactose, maltitol, guanosine, glyceryl starch, Triticum vulgare starch, trehalose, sucrose and its derivatives, raffinose or sodium chondroitin sulfate.

Use may also be made, as surface treatment agents, of C₁-C₂₀ alkylene glycols or C₁-C₂₀ alkylene glycol ethers, alone or used in combination with tri (C₁-C₂₀)alkylsilanes.

Mention may be made, as examples, of the pigments surface-treated with PEG alkyl ether alkoxysilane, such as, for example, the pigments treated with PEG-8 methyl ether triethoxysilane which are sold by Kobo under the name SW pigments.

Silicones, such as dimethicones having hydrophilic groups, also known under the name dimethicone copolyols or alkyl dimethicone copolyols, may also be suitable for the invention as surface treatment agents. In particular, such dimethicones can comprise, as repeat units, C₁-C₂₀ alkylene oxides, such as ethylene or propylene oxides.

Mention may be made, as example, of the pigment treated with PEG-12 dimethicone, sold by Sensient Corporation under the name LCW AQ® Pigment.

The amount of pigments coated with at least one hydrophilic compound and/or of uncoated pigments is in particular conditioned by the intended use of the cosmetic composition under consideration, and the adjustment of this amount obviously falls within the competence of the formulator of the composition.

### Nonemulsifying Organopolysiloxane Elastomer

According to a preferred form of the invention, the composition additionally comprises at least one nonemulsifying organopolysiloxane elastomer.

The term “nonemulsifying” defines organopolysiloxane elastomers not containing a hydrophilic chain and in particular not containing polyoxyalkylene units (in particular polyoxyethylene or polyoxypropylene units) or polyglyceryl units.

Thus, the organopolysiloxane elastomer can be obtained by an addition-crosslinking reaction of diorganopolysiloxane containing at least one hydrogen bonded to silicon and of diorganopolysiloxane having ethylenically unsaturated groups bonded to silicon, in particular in the presence of a platinum catalyst; or by condensation-crosslinking-dehydrogenation reaction between a hydroxyl-terminated diorganopolysiloxane and a diorganopolysiloxane containing at least one hydrogen bonded to silicon, in particular in the presence of an organotin compound; or by a condensation-crosslinking reaction of a hydroxyl-terminated diorganopolysiloxane and of a hydrolyzable organopolysiloxane; or by thermal crosslinking of organopolysiloxane, in particular in the presence of an organoperoxide catalyst; or by crosslinking of organopolysiloxanes by high-energy radiation, such as gamma rays, ultraviolet rays or an electron beam.

Preferably, the organopolysiloxane elastomer is obtained by an addition-crosslinking reaction (A) of diorganopolysiloxane containing at least two hydrogens each bonded to a silicon and (B) of diorganopolysiloxane having at least two ethylenically unsaturated groups bonded to silicon, in particular in the presence (C) of a platinum catalyst.

In particular, the organopolysiloxane elastomer can be obtained by reaction of dimethylvinylsiloxy-terminated dimethylpolysiloxane and of trimethylsiloxy-terminated methylhydropolysiloxane, in the presence of a platinum catalyst.

The compound (A) is the base reactant for the formation of organopolysiloxane elastomer and the crosslinking is carried out by an addition reaction of the compound (A) with the compound (B) in the presence of the catalyst (C).

The compound (A) is in particular an organopolysiloxane having at least two hydrogen atoms bonded to separate silicon atoms in each molecule.

The compound (A) can exhibit any molecular structure, in particular a linear-chain or branched-chain structure or a cyclic structure.

The compound (A) can have a viscosity at 25° C. ranging from 1 to 50 000 centistokes, in particular in order to be readily miscible with the compound (B).

The organic groups bonded to the silicon atoms of the compound (A) can be alkyl groups, such as methyl, ethyl, propyl, butyl or octyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbon groups, such as an epoxy group, a carboxylate ester group or a mercapto group.

The compound (A) can thus be chosen from trimethylsiloxy-terminated methylhydropolysiloxanes, trimethylsiloxy-terminated dimethylsiloxane/methylhydrosiloxane copolymers or cyclic dimethylsiloxane/methylhydrosiloxane copolymers.

The compound (B) is advantageously a diorganopolysiloxane having at least two lower (for example C₂-C₄)alkenyl groups; the lower alkenyl group can be chosen from vinyl, allyl and propenyl groups. These lower alkenyl groups can be located at any position of the organopolysiloxane molecule but are preferably located at the ends of the organopolysiloxane molecule. The organopolysiloxane (B) can have a branched-chain, linear-chain, cyclic or network structure but the linear-chain structure is preferred. The compound (B) can have a viscosity ranging from the liquid state to the gum state. Preferably, the compound (B) has a viscosity of at least 100 centistokes at 25° C.

In addition to the abovementioned alkenyl groups, the other organic groups bonded to the silicon atoms in the compound (B) can be alkyl groups, such as methyl, ethyl, propyl, butyl or octyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbon groups, such as an epoxy group, a carboxylate ester group or a mercapto group.

The organopolysiloxanes (B) can be chosen from methylvinylpolysiloxanes, methylvinylsiloxane/dimethylsiloxane copolymers, dimethylvinylsiloxy-terminated dimethylpolysiloxanes, dimethylvinylsiloxy-terminated dimethylsiloxane/methylphenylsiloxane copolymers, dimethylvinylsiloxy-terminated dimethylsiloxane/diphenylsiloxane/methylvinylsiloxane copolymers, trimethylsiloxy-terminated dimethylsiloxane/methylvinylsiloxane copolymers, trimethylsiloxy-terminated dimethylsiloxane/methylphenylsiloxane/methylvinylsiloxane copolymers, dimethylvinylsiloxy-terminated methyl (3,3,3-trifluoropropyl) polysiloxanes and dimethylvinylsiloxy-terminated dimethylsiloxane/methyl (3,3,3-trifluoropropyl) siloxane copolymers.

In particular, the organopolysiloxane elastomer can be obtained by reaction of dimethylvinylsiloxy-terminated dimethylpolysiloxane and of trimethylsiloxy-terminated methylhydropolysiloxane, in the presence of a platinum catalyst.

According to another alternative form, the compound (B) can be an unsaturated hydrocarbon compound having at least two lower (for example C₂-C₄)alkenyl groups; the lower alkenyl group can be chosen from vinyl, allyl and propenyl groups. These lower alkenyl groups can be located at any position of the molecule but are preferably located at the ends. Mention may be made, by way of example, of hexadiene and in particular of 1,5-hexadiene.

Advantageously, the sum of the number of ethylenic groups per molecule of the compound (B) and of the number of hydrogen atoms bonded to silicon atoms per molecule of the compound (A) is at least 5.

It is advantageous for the compound (A) to be added in an amount such that the molecular ratio of the total amount of hydrogen atoms bonded to the silicon atoms in the compound (A) to the total amount of all the ethylenically unsaturated groups in the compound (B) is within the range from 1.5/1 to 20/1.

The compound (C) is the catalyst of the crosslinking reaction and is in particular chloroplatinic acid, chloroplatinic acid-olefin complexes, chloroplatinic acid-alkenylsiloxane complexes, chloroplatinic acid-diketone complexes, platinum black and platinum on a support.

The catalyst (C) is preferably added from 0.1 to 1000 parts by weight and better still from 1 to 100 parts by weight, as clean platinum metal, per 1000 parts by weight of the total amount of the compounds (A) and (B).

Use may be made, for example, as spherical nonemulsifying elastomers, of those sold under the names DC 9040®, DC 9041®, DC 9509® and DC 9505® by Dow Corning.

Use may also be made of those sold under the names KSG-6®, KSG-15®, KSG-16®, KSG-18®, KSG-41®, KSG-42®, KSG-43® and KSG-44® by Shin-Etsu; Gransil SR 5CYC® gel, Gransil SR DMF 10 gel®, Gransil SR DC556 gel®, Gransil RPS®, Gransil DMG-6® and Gransil DMG-6® LC from Grant Industries; 1229-02-167®, 1229-02-168® and SFE 839® from General Electric.

According to a preferred embodiment, the composition according to the invention comprises at least one nonemulsifying organopolysiloxane elastomer in the gel form conveyed in at least one silicone oil, preferably a linear silicone oil of the dimethicone type.

Mention may be made, for example, of the Polysilicone-11/Dimethicone mixture, such as the commercial products Gransil DMG-6® and Gransil DMG-6R LC from Grant Industries.

The composition according to the invention comprises a content of organopolysiloxane elastomer, expressed as organopolysiloxane elastomer (i.e., as active material), preferably varying from 0.1% to 10% by weight, preferably from 0.5% to 5% by weight, with respect to the weight of the composition.

According to a specific form of the invention, the composition comprises:
a) at least one oily continuous phase comprising at least one dimethicone with a viscosity of from 10⁻⁶ to 8×10⁻⁶ m²/s and/or a C₈-C₁₆ isoalkane, preferably isododecane, and
b) at least one aqueous phase dispersed in said oily phase, comprising at least one water-soluble organic UV screening agent chosen from Terephthalylidene Dicamphor, Phenyl Benzimidazole Sulfonic Acid, 1,4-bis-benzimidazolyl-phenylene-3,3′,5,5′-tetrasulfonic acid and their mixtures; and more particularly Phenylbenzimidazole Sulfonic Acid; and
c) sodium hydroxide; and
d) at least one hydrophobic film-forming polymer chosen from trimethylsiloxysilicate resins, and
e) at least the emulsifying surfactant PEG/PPG-18/18 Dimethicone, preferably in the form of a mixture with a dimethicone, and
f) niacinamide.

According to a specific form of the invention, the composition comprises:
a) at least one oily continuous phase comprising at least one dimethicone with a viscosity of from 10⁻⁶ to 8×10⁻⁶ m²/s and/or a C₈-C₁₆ isoalkane, preferably isododecane, and
b) at least one aqueous phase dispersed in said oily phase, comprising at least one water-soluble organic UV screening agent chosen from Terephthalylidene Dicamphor, Phenyl Benzimidazole Sulfonic Acid, 1,4-bis-benzimidazolyl-phenylene-3,3′,5,5′-tetrasulfonic acid and their mixtures; and more particularly Phenylbenzimidazole Sulfonic Acid; and
c) sodium hydroxide; and
d) at least one hydrophobic film-forming polymer chosen from trimethylsiloxysilicate resins, and
e) at least the mixture of emulsifying surfactants PEG/PPG-18/18 Dimethicone and PEG-10 Dimethicone, preferably the mixture of emulsifying surfactants: Dimethicone and PEG/PPG-18/18 Dimethicone and PEG-10 Dimethicone,
f) niacinamide.

According to a specific embodiment, the composition additionally comprises at least one pigment chosen from titanium dioxides and/or iron oxides, in particular coated with a hydrophobic surface treatment agent, especially with an N-acylated amino acid and/or one of its salts, in particular with a glutamic acid derivative and/or one of its salts, especially a stearoyl glutamate, such as, for example, aluminum stearoyl glutamate.

### Additives

The compositions according to the invention can additionally comprise additives commonly used in care and/or makeup products, such as organic UV screening agents other than those described above; inorganic UV screening agents; moisturizing agents, such as polyols, for example glycerol, propanediol or pentylene glycol; fillers; colorants; thickening or gelling agents; preservatives; chelating agents; fragrances; and their mixtures.

### Fillers

The compositions in accordance with the invention can also comprise at least one filler, of organic or inorganic nature, which makes it possible in particular to confer on them additional properties of improved stability, wear property, coverage and/or mattness.

The term “filler” should be understood as meaning colorless or white solid particles of any form which are provided in an insoluble form dispersed in the medium of the composition. These particles, of inorganic or organic nature, make it possible to confer body or firmness on the composition and/or softness and uniformity on the makeup.

The fillers used in the compositions according to the present invention can be of lamellar, globular, spherical or fibrous forms or of any other form intermediate between these defined forms.

The fillers according to the invention may or may not be surface-coated, and in particular they may be surface-treated with silicones, amino acids, fluorinated derivatives or any other substance which promotes the dispersion and the compatibility of the filler in the composition.

Mention may be made, as examples of inorganic fillers, of talc, mica, silica, hollow silica microspheres, kaolin, calcium carbonate, magnesium carbonate, hydroxyapatite, boron nitride, glass or ceramic microcapsules, composites of silica and of titanium dioxide, such as the TSG® series sold by Nippon Sheet Glass, or hydrophobic silica aerogels.

Mention may be made, as examples of organic fillers, of powders formed of polyamide (Nylon® Orgasol from Atochem), of polyethylene, of polymethyl methacrylate, of polytetrafluoroethylene (Teflon®) or of acrylic acid copolymers (Polytrap® from Dow Corning), lauroyl lysine, hollow polymeric microspheres, such as those of polyvinylidene chloride/acrylonitrile, for example Expancel® (Nobel Industrie), Hexamethylene Diisocyanate/Trimethylol Hexyllactone copolymer powder (Plastic Powder® from Toshiki), silicone resin microbeads (Tospearls® from Toshiba, for example), synthetic or natural micronized waxes, metal soaps derived from organic carboxylic acids having from 8 to 22 carbon atoms, preferably from 12 to 18 carbon atoms, for example zinc stearate, magnesium stearate, lithium stearate, zinc laurate or magnesium myristate, Polypore® L 200 (Chemdal Corporation), or powders formed of crosslinked elastomeric organopolysiloxane coated with silicone resin, in particular with silsesquioxane resin, as described, for example, in the patent U.S. Pat. No. 5,538,793. It can also be a cellulose powder, such as that sold by Daito in the Cellulobeads range.

### Silica Particles

According to a preferred form, the composition according to the invention additionally comprises silica particles chosen from hydrophobic silica aerogel particles, silica particles other than the preceding ones, and their mixtures.

i. Hydrophobic Silica Aerogels

Hydrophobic silica aerogels are porous materials obtained by replacing (in particular by drying) the liquid component of a silica gel with air. They are generally synthesized by a sol-gel process in a liquid medium and then dried, usually by extraction with a supercritical fluid, the one most commonly used being supercritical CO₂. This type of drying makes it possible to avoid contraction of the pores and of the material. The sol-gel process and the various drying operations are described in detail in Brinker C. J. and Scherer G. W., Sol-Gel Science, New York, Academic Press, 1990.

The hydrophobic silica aerogels used according to the present invention are preferably silylated silica aerogels (INCI name: Silica Silylate).

The term “hydrophobic silica” is understood to mean any silica, the surface of which is treated with silylating agents, for example with halogenated silanes, such as alkylchlorosilanes, siloxanes, in particular dimethylsiloxanes, such as hexamethyldisiloxane, or silazanes, so as to functionalize the OH groups with Si—Rₙ silyl groups, for example trimethylsilyl groups.

As regards the preparation of hydrophobic silica aerogel particles which have been surface-modified by silylation, reference may be made to the document U.S. Pat. No. 7,470,725.

Use will be made in particular of aerogel particles formed of hydrophobic silica which is surface-modified with trimethylsilyl groups (trimethylsiloxylated silica).

The term “hydrophobic aerogel particles” is understood to mean any particle of the aerogel type exhibiting a water absorption capacity at the wet point of less than 0.1 ml/g, i.e. less than 10 g of water per 100 g of particle.

The absorption capacity, measured at the wet point and denoted WP, corresponds to the amount of a solvent (expressed in grams or in milliliters) which it is necessary to add to 1 g of particles in order to obtain a homogeneous paste. It is measured according to the “wet point” method or the method for determining the uptake of solvent (water or oil) of a powder described in the standard NF T 30-022. It corresponds to the amount of solvent adsorbed onto the available surface of the powder and/or absorbed by the powder by measurement of the wet point, described below:

A glass plate (25×25 mm) is deposited on a balance, an amount w of 1 g of powder is weighed out onto the glass plate and then a solvent (water or isononyl isononanoate, for example) is added dropwise. The solvent is added gradually to the powder, everything being regularly kneaded (every 3 to 4 drops) using a spatula. The addition of solvent is halted when a homogeneous paste is obtained. This paste must be able to be spread over the glass plate without cracks or the formation of lumps. The weight of solvent necessary to obtain the wet point is recorded. The mean over three tests will be taken. As the density of the solvent is known, the volume Vs (expressed in ml) of solvent used is deduced therefrom. The solvent uptake corresponds to the ratio Vs/w.

Preferably, the hydrophobic silica aerogel particles according to the invention preferably have an oil absorption capacity, measured at the wet point, ranging from 5 to 18 ml/g, preferably from 6 to 15 ml/g and better still from 8 to 12 ml/g.

The hydrophobic silica aerogel particles used in the present invention preferably exhibit a specific surface per unit of weight (S_{W}) ranging from 200 to 1500 m²/g, preferably from 600 to 1200 m²/g and better still from 600 to 800 m²/g, and a size, expressed as volume-average diameter (D[0.5]), of less than 1500 μm and preferably ranging from 1 to 30 μm, preferably from 5 to 25 μm, better still from 5 to 20 μm and even better still from 5 to 15 μm.

The specific surface per unit of weight can be determined by the nitrogen absorption method, known as the BET (Brunauer-Emmett-Teller) method, described in The Journal of the American Chemical Society, Vol. 60, page 309, February 1938, and corresponding to the international standard ISO 5794/1 (Annex D). The BET specific surface corresponds to the total specific surface of the particles under consideration.

The sizes of the aerogel particles according to the invention can be measured by static light scattering using a commercial particle size analyser of Mastersizer 2000® type from Malvern. The data are processed on the basis of the Mie scattering theory. This theory, which is exact for isotropic particles, makes it possible to determine, in the case of nonspherical particles, an effective particle diameter. This theory is in particular described in the publication by Van de Hulst, H. C., Light Scattering by Small Particles, Chapters 9 and 10, Wiley, New York, 1957.

The hydrophobic silica aerogel particles used in the present invention can advantageously exhibit a tamped density ranging from 0.02 g/cm³ to 0.10 g/cm³ and preferably from 0.02 g/cm³ to 0.08 g/cm³.

In the context of the present invention, this density can be assessed according to the following protocol, known as the tamped density protocol: 40 g of powder are poured into a graduated measuring cylinder and then the measuring cylinder is placed on a Stav 2003® device from Stampf Volumeter; the measuring cylinder is subsequently subjected to a series of 2500 tamping actions (this operation is repeated until the difference in volume between two consecutive tests is less than 2%) and then the final volume Vf of tamped powder is measured directly on the measuring cylinder. The tamped density is determined by the ratio w/Vf, in the case in point 40/Vf (Vf being expressed in cm³ and w in g).

According to one embodiment, the hydrophobic aerogel particles used in the present invention exhibit a specific surface per unit of volume (S_{V}) ranging from 5 to 60 m²/cm³, preferably from 10 to 50 m²/cm³ and better still from 15 to 40 m²/cm³.

The specific surface per unit of volume is given by the relationship: S_{V}=S_{W}×ρ, where ρ is the tamped density, expressed in g/cm³, and S_{W} is the specific surface per unit of weight, expressed in m²/g, as defined above.

According to a specific embodiment, the aerogel particles used are inorganic and more particularly hydrophobic silica aerogel particles exhibiting the properties stated above.

Mention may be made, as hydrophobic silica aerogels which can be used in the invention, for example, of the aerogel sold under the name VM-2260 (INCI name: Silica Silylate) by Dow Corning, the particles of which exhibit an average size of approximately 1000 microns and a specific surface per unit of weight ranging from 600 to 800 m²/g.

Mention may also be made of the aerogels sold by Cabot under the references Aerogel TLD 201®, Aerogel OGD 201®, Aerogel TLD 203®, Enova® Aerogel MT 1100 and Enova Aerogel MT 1200®.

Use will more particularly be made of the aerogel sold under the name VM-2270® (INCI name: Silica Silylate) by Dow Corning, the particles of which exhibit an average size ranging from 5 to 15 microns and a specific surface per unit of weight ranging from 600 to 800 m²/g.

Use will also be made of the aerogel sold under the name Enova® Aerogel MT 1100 (INCI name: Silica Silylate) by Cabot, the particles of which exhibit an average size ranging from 2 to 25 microns and a specific surface per unit of weight ranging from 600 to 800 m²/g.

The hydrophobic aerogel particles represent from 0.05% to 10% by weight, preferably from 0.1% to 8% by weight, better still from 0.2% to 5% by weight and more preferably from 0.3% to 3% by weight, with respect to the total weight of the composition.

### Other Silica Particles

The other silicas which can be used can be natural and untreated. Mention may thus be made of the silicas provided under the names Sillitin N85®, Sillitin N87®, Sillitin N82®, Sillitin V85® and Sillitin V88® by Hoffmann Mineral.

They can be fumed silicas.

The fumed silicas can be obtained by high-temperature pyrolysis of a volatile silicon compound in an oxyhydrogen flame, producing a finely divided silica. This process makes it possible in particular to obtain hydrophilic silicas which exhibit a large number of silanol groups at their surface. It is possible to chemically modify the surface of said silica by chemical reaction generating a reduction in the number of silanol groups. It is possible in particular to replace silanol groups by hydrophobic groups: a hydrophobic silica is then obtained.

The hydrophobic groups can be:
(a) trimethylsiloxy groups, which are in particular obtained by treatment of fumed silica in the presence of hexamethyldisilazane. Silicas thus treated are known as Silica Silylate according to the CTFA (6th Edition, 1995);
(b) dimethylsilyloxy or polydimethylsiloxane groups, which are in particular obtained by treatment of fumed silica in the presence of polydimethylsiloxane or dimethyldichlorosilane. Silicas thus treated are known as Silica Dimethyl Silylate according to the CTFA (6th Edition, 1995).

Mention may more particularly be made, as silica powders other than silicon aerogels, of:
the porous silica microspheres sold under the names Silica Beads SB-700® by Miyoshi and Sunsphere® H51 and Sunsphere® H33 by Asahi Glass;
the polydimethylsiloxane-coated amorphous silica microspheres sold under the names SA Sunsphere® H33® and SA Sunsphere® H53® by AGC Sitech;
precipitated silica microspheres, for example coated with mineral wax, such as polyethylene, and sold in particular under the name Acematt® OK 412® by Evonik Degussa.

Use will more particularly be made, as silica powder, of porous silica microspheres, such as those sold under the names Silica Beads SB-700® by Miyoshi and Sunsphere® H51 and Sunsphere® H33 by AGC Sitech.

The silica particles other than the hydrophobic silica aerogel particles are present in the composition according to the invention in a content ranging from 0.01% to 15% by weight, preferably ranging from 0.1% to 10% by weight and very preferentially ranging from 0.5% to 5% by weight, with respect to the total weight of the composition.

According to a preferential form, the composition according to the invention will comprise a mixture comprising at least hydrophobic silica aerogel particles, such as those described above, and other silica particles, such as those described above, in particular porous silica microspheres.

### Additional Colorants

A composition according to the invention can additionally comprise at least one additional colorant, preferably in a proportion of at least 0.01% by weight, with respect to the total weight of the composition.

For obvious reasons, this amount is liable to vary significantly from the viewpoint of the desired intensity of the color effect and of the color intensity provided by the colorants under consideration, and its adjustment clearly falls within the competence of a person skilled in the art.

The additional colorants suitable for the invention can be water-soluble but also fat-soluble.

Within the meaning of the invention, the term “water-soluble colorant” is understood to mean any natural or synthetic, generally organic, compound which is soluble in an aqueous phase or water-miscible solvents and which is capable of imparting color.

Mention may in particular be made, as water-soluble dyes suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocyanin, black carrot, hibiscus or elder), caramel or riboflavin.

The water-soluble dyes are, for example, beetroot juice and caramel.

Within the meaning of the invention, the term “fat-soluble colorant” is understood to mean any natural or synthetic, generally organic, compound which is soluble in an oily phase or solvents miscible with a fatty substance and which is capable of imparting color.

Mention may in particular be made, as fat-soluble dyes suitable for the invention, of synthetic or natural fat-soluble dyes, such as, for example, DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto or curcumin.

### Cosmetic Compositions

The present invention also relates to a cosmetic composition comprising, in a physiologically acceptable medium, a composition as defined above.

The term “physiologically acceptable medium” is understood to denote a medium which is particularly suitable for the application of a composition of the invention to the skin.

The physiologically acceptable medium is generally suited to the nature of the support onto which the composition has to be applied, and also to the appearance under which the composition has to be packaged.

### Applications

According to one embodiment, a composition of the invention can advantageously be provided in the form of a composition for caring for the skin of the body or of the face, in particular of the face.

According to another embodiment, a composition of the invention can advantageously be provided in the form of a composition for making up keratin materials, in particular the skin of the body or of the face, in particular of the face.

Thus, according to a sub-mode of this embodiment, a composition of the invention can advantageously be provided in the form of a base composition for makeup.

A composition of the invention can advantageously be provided in the form of a foundation.

According to another sub-mode of this embodiment, a composition of the invention can advantageously be provided in the form of a composition for making up the skin and in particular the face. It can thus be an eyeshadow or a face powder.

Such compositions are in particular prepared according to the general knowledge of a person skilled in the art.

Throughout the description, including the claims, the expression “comprising a” should be understood as being synonymous with “comprising at least one”, unless otherwise specified.

The expressions “of between . . . and . . . ” and “ranging from . . . to . . . ” should be understood as meaning limits included, unless otherwise specified.

The invention is illustrated in greater detail by the examples and figures presented below. Unless otherwise indicated, the amounts indicated are expressed as percentages by weight.

### EXAMPLES 1 AND 2 (INVENTION) AND EXAMPLE 1A* (OUTSIDE THE INVENTION)

Compositions 1 and 2 according to the invention and compositions 1a and 1b outside the invention were produced.

TABLE 1

| Phase | Ingredients | Ex1 | Ex2 | 1a* | 1b* |
|---|---|---|---|---|---|
| A1 | Dimethicone (and) | 10.0 (2.5% | 10.0 (2.5% | 10.0 (2.5% | 10.0 (2.5% |
| | PEG/PPG-18/18 | as | as | as | as |
| | Dimethicone | active | active | active | active |
| | (25% by weight of | material) | material) | material) | material) |
| | active material) (X-22- | | | | |
| | 6711D ® - Shin Etsu) | | | | |
| | Isododecane | 3.7 | 3.7 | 3.7 | 3.7 |
| | PEG-10 Dimethicone | 0.65 | 0.65 | 0.65 | 0.65 |
| | (KF-6017 ® - Shin Etsu) | | | | |
| A2 | Trimethylsiloxysilicate | 6.0 | 6.0 | 6.0 | 6.0 |
| | and Isododecane (75% | | | | |
| | by weight of active | | | | |
| | material) | | | | |
| | (Silsoft 74 ® - | | | | |
| | Momentive | | | | |
| | Performance | | | | |
| | Materials) | | | | |
| A3 | Dimethicone (and) | 7.9 | 7.9 | 7.9 | 7.9 |
| | Polysilicone-11 (16% | | | | |
| | by weight of active | | | | |
| | material) | | | | |
| | (Gransil DMG-6 ® - | | | | |
| | Grant Industries) | | | | |
| A4 | Silica | 1.5 | 1.5 | 1.5 | 1.5 |
| | (Sunsphere H 51 ® - | | | | |
| | AGC Si-Tech) | | | | |
| | Silica Silylate | 0.5 | 0.5 | 0.5 | 0.5 |
| | (Dow Corning VM- | | | | |
| | 2270 ® Aerogel Fine | | | | |
| | Particles - Dow | | | | |
| | Corning) | | | | |
| B1 | Water | q.s. | q.s. | q.s. | q.s. |
| | | for 100 | for 100 | for 100 | for 100 |
| | Glycerin | 2.5 | 2.5 | 2.5 | 2.5 |
| | Propylene Glycol | 3.5 | 3.5 | 3.5 | 3.5 |
| | Sodium Chloride | 0.7 | 0.7 | 0.7 | 0.7 |
| | Phenoxyethanol | 0.7 | 0.7 | 0.7 | 0.7 |
| | Phenylbenzimidazole | 2.70 | 2.70 | 2.70 | 2.70 |
| | Sulfonic Acid | | | | |
| | (Eusolex 232 ® - | | | | |
| | Merck) | | | | |
| | Sodium Hydroxide | 0.45 | 0.45 | 0.45 | 0.45 |
| | Niacinamide | 2.0 | 0.11 | — | 2.0 |
| C | Propylene Glycol | — | — | — | 15.27 |
| | Dicaprylate/Dicaprate | | | | |
| | Dimethicone | 15.27 | 15.27 | 15.27 | — |
| | (Dowsil SH 200 ® C | | | | |
| | Fluid 5 cSt Dow | | | | |
| | Corning) | | | | |
| D | Alcohol Denat. | 5.0 | 5.0 | 5.0 | 5.0 |

### a) Preparation of the Aqueous Phase:

All the ingredients of phase B1 were weighed out and stirring was carried out with a Rayneri mixer (deflocculator). The pH was adjusted with sodium hydroxide and the amount of water lost was added.

### b) Preparation of the Oily Phase:

The nonvolatile dimethicone of phase C and then the isododecane and the surfactants of phase A1 were weighed out and mixed under a Moritz mixer (rotor/stator) for 5 min while adjusting the speed in order to have a vortex and then the assembly was placed under a bath of ice-cold water. The silicone elastomer of phase A3 was weighed out and mixing was carried out for 15 minutes while adjusting the speed in order to always have a vortex. The film-forming polymer of phase A3 and the fillers of phase A4 were subsequently weighed out. It is possible to disperse the Dow Corning VM-2270® Aerogel Fine Particles—Dow Corning in isododecane beforehand to facilitate the processing. Mixing was then carried out for 15 minutes while adjusting the speed in order to always have a vortex.

### c) Emulsification

The emulsion was produced under a Moritz mixer and still under a bath of ice-cold water, the aqueous phase being introduced into the fatty phase. The assembly was left at the speed of 1300 rev/min for 20 min (gradual increase/vortex).

Subsequently, the alcohol was added under a Rayneri mixer and dispersing was allowed to take place for 5 min while keeping the temperature below 40° C.

### Haze Measurements

Examples 1 and 2 according to the invention with niacinamide and counterexamples 1a and 1b were subjected to haze performance measurements after spreading in the form of films, according to the following protocol:

### a) Spreading Protocol

Each test composition was spread using an automatic spreader in the form of a film with a thickness of 25 μm over a transparent polyester sheet. The deposit obtained was left at ambient temperature (25° C.) for 1 hour.

### b) Haze Measurement Protocol

The haze measurements were carried out using the Haze Gard® device (Haze-Gard Plus Brant Industrie S.A.R.L.—BYK Gardner).

The laser was switched on 1 hour before the measurements. After a phase of calibrating the appliance, the film, dried beforehand for 1 hour, was positioned in the appliance in the path of the laser. The haze measurements were carried out automatically by the appliance.

Each composition was spread three times and 10 haze measurements were carried out per composition in order to determine a haze mean and a standard deviation. The results are shown in the table below.

### Stability Tests

Examples 1 and 1b were introduced in a proportion of 20 g into a glass sample tube. Each sample tube was placed in a cycle oven of the Heraeus Voetsch® type for 10 days. Every 24 hours, each sample tube was subjected to the following 4 temperature cycles of 6 hours.
6 hours at +20° C.
6 hours of gradual fall in temperature from 20° C. to −20° C.
6 hours at −20° C.
6 hours of gradual rise in temperature from −20° C. to +20° C.

After 10 days, the appearance of each example 1 and 1b was observed:
i) macroscopically with the naked eye; and
ii) microscopically with the Leica DM2500® microscope with a X10 magnification.
The results obtained are shown in tables 2 and 3 below.

TABLE 2

| | | | | Example 1b |
|---|---|---|---|---|
| | | | | (outside the |
| | | | Example 1a | invention) |
| | Example 1 | Example 2 | (outside the | 2% |
| | (invention) | (invention) | invention) | Niacinamide |
| | 2% | 0.11% | 0% | 15.27% |
| | Niacinamide | Niacinamide | Niacinamide | Propylene |
| | 15.27% | 15.27% | 15.27% | Glycol |
| Haze | Dimethicone | Dimethicone | Dimethicone | Dicaprylate/ |
| measurements | 5 cSt | 5 cSt | 5 cSt | Dicaprate |
| Mean over 3 | 63.20 | 54.90 | 47.83 | 35.61 |
| tests | | | | |
| Standard | 0.29 | 0.91 | 0.87 | 0.11 |
| deviation over | | | | |
| 3 tests | | | | |

The haze tests have shown that examples 1 and 2 according to the invention comprising niacinamide, isododecane and a volatile dimethicone (5 cSt) produced better blurring of imperfections, in comparison
with example 1a outside the invention of identical composition but without niacinamide and
with example 1b outside the invention of identical composition but comprising, at the same concentration, a nonvolatile ester oil, Propylene Glycol Dicaprylate/Dicaprate, in place of the volatile dimethicone (5 cSt).

TABLE 3

| | | Example 1b |
|---|---|---|
| | | (outside the invention) |
| | Example 1 | 2% |
| Stability in the | (invention) | Niacinamide |
| oven −20° C.-+20° | 2% Niacinamide | 15.27% |
| C. cycle after | 15.27% Dimethicone | Propylene Glycol |
| 10 days | 5 cSt | Dicaprylate/Dicaprate |
| Macroscopic | Stable | Slightly glossy surface |
| appearance | Homogeneous | |
| Microscopic | Stable | Fragile emulsion with less |
| appearance | Homogeneous and | homogeneous foundation, |
| | regular foundation, | slightly slack edges and |
| | clean edges | small crystals visible |

The tests of stability on storage for 10 days have shown that example 1 according to the invention comprising niacinamide, isododecane and a volatile dimethicone (5 cSt) has remained stable and homogeneous, unlike example 1b.

1. A composition in the form of a water-in-oil emulsion, in particular comprising a physiologically acceptable medium, especially for coating keratin materials, more particularly for making up and/or caring for keratin materials, comprising:
a) at least one oily continuous phase comprising at least one volatile oil chosen from volatile hydrocarbon oils, volatile silicone oils and their mixtures, and
b) at least one aqueous phase dispersed in said oily phase, comprising at least one water-soluble organic UV screening agent comprising at least one benzylidenecamphor sulfonic acid group and/or at least one water-soluble organic UV screening agent comprising at least one benzoxazole sulfonic acid group, and
c) at least one organic or inorganic base capable of partially or completely neutralizing said water-soluble organic UV screening agent(s), and
d) at least one hydrophobic film-forming polymer, and
e) at least one emulsifying surfactant chosen from polyoxyalkylenated linear polydimethylmethylsiloxanes with an HLB≥8.0, and
f) at least one vitamin B3;
said composition not containing a nonvolatile ester oil.

2. The composition as claimed in claim 1, where the volatile hydrocarbon oil or oils are chosen from C₈-C₁₆ isoalkanes of petroleum origin, in particular, the volatile hydrocarbon oil is isododecane.

3. The composition as claimed in claim 1, where the volatile silicone oil or oils are chosen from linear silicone oils with a viscosity ranging from 10⁻⁶ to 8×10⁻⁶ m²/s, in particular chosen from octamethyltrisiloxane, decamethyltetrasiloxane, dodecamethylpentasiloxane and their mixtures, and more particularly dodecamethylpentasiloxane.

4. The composition as claimed in any one of claims 1 to 3, comprising a mixture of at least one volatile hydrocarbon oil and of at least one volatile silicone oil, in particular a mixture of isododecane and of dodecamethylpentasiloxane.

5. The composition as claimed in any one of the preceding claims, where the total concentration of oily phase varies from 20% to 95% by weight and more particularly varies from 30% to 60% by weight, with respect to the total weight of the composition.

6. The composition as claimed in any one of the preceding claims, where the volatile oil(s) are present at concentrations ranging from 5% to 45% by weight, more preferentially ranging from 10% to 40% by weight and more preferentially still ranging from 12% to 35% by weight, with respect to the total weight of the composition.

7. The composition as claimed in any one of the preceding claims, where the aqueous phase is present in a concentration of at least 20% by weight, preferably ranging from 30% to 60% by weight and more particularly from 35% to 50% by weight, with respect to the total weight of said composition.

8. The composition as claimed in any one of the preceding claims, in which the water-soluble UV screening agent is chosen from Terephthalylidene Dicamphor Sulfonic Acid, 1,4-bis-benzimidazolyl-phenylene-3,3′,5,5′-tetrasulfonic acid, Phenylbenzimidazole Sulfonic Acid and their mixtures, and more particularly Phenylbenzimidazole Sulfonic Acid.

9. The composition as claimed in any one of the preceding claims, where the water-soluble UV screening agent(s) are present in the composition of the invention at concentrations ranging from 0.1% to 10% by weight, more preferentially ranging from 1% to 8% by weight and more preferentially still ranging from 2% to 5% by weight, with respect to the total weight of the composition.

10. The composition as claimed in any one of the preceding claims, where the organic base is chosen from amino acids, alkanolamines and their mixtures.

11. The composition as claimed in any one of the preceding claims, where the inorganic base is chosen from alkali metal cation bases and alkaline earth metal cation bases, and more particularly is sodium hydroxide.

12. The composition as claimed in any one of the preceding claims, where the hydrophobic film-forming polymer is chosen from:
silicone resins;
block ethylenic copolymers;
vinyl polymers comprising at least one unit derived from carbosiloxane dendrimer;
silicone-acrylate copolymers;
their mixtures; and more particularly chosen from a trimethylsiloxysilicate resin.

13. The composition as claimed in any one of the preceding claims, comprising from 0.5% to 15% by weight, more preferentially from 1% to 10% by weight, more particularly from 2% to 7% by weight, as active material of hydrophobic film-forming polymer(s), with respect to the total weight of the composition.

14. The composition as claimed in any one of the preceding claims, where the linear polyoxyalkylenated polydimethylmethylsiloxane emulsifying surfactants correspond to the following formula (I): 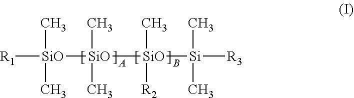
in which R₁, R₂ and R₃, independently of one another, represent a C₁-C₆ alkyl radical or a —(CH₂)ₓ—(OCH₂CH₂)_{y}—(OCH₂CH₂CH₂)_{z}—OR₄ radical, at least one R₁, R₂ or R₃ radical not being an alkyl radical, R₄ being a hydrogen, a C₁-C₃ alkyl radical or a C₂-C₄ acyl radical;
A is an integer ranging from 0 to 200;
B is an integer ranging from 0 to 50; provided that A and B are not simultaneously equal to zero;
x is an integer ranging from 0 to 6;
y is an integer ranging from 1 to 30;
z is an integer ranging from 0 to 30.

15. The composition as claimed in claim 14, where the linear polyoxyalkylenated polydimethylmethylsiloxane emulsifying surfactants of formula (I) are chosen from those for which R₁=R₃=methyl radical, x=0 to 3 and R₄ is a hydrogen.

16. The composition as claimed in any one of the preceding claims, where the linear polyoxyalkylenated polydimethylmethylsiloxane emulsifying surfactant is chosen from:
PEG/PPG-8/8 Dimethicone;
PEG/PPG-18/18 Dimethicone;
the Cyclopentasiloxane and PEG/PPG-18/18 Dimethicone mixture;
the Cyclotetrasiloxane and Cyclopentasiloxane and PEG/PPG-18/18 Dimethicone mixture;
the Dimethicone and PEG/PPG-18/18 Dimethicone mixture;
the PEG/PPG-19/19 Dimethicone and C₁₃-C₁₆ Isoparaffin and C₁₀-C₁₃ Isoparaffin mixture;
PEG-3 Dimethicone;
PEG-10 Dimethicone;
their mixtures.

17. The composition as claimed in any one of the preceding claims, comprising PEG/PPG-18/18 Dimethicone, preferably in combination with a silicone oil and more particularly a silicone oil of the dimethicone type.

18. The composition as claimed in any one of the preceding claims, comprising a mixture of PEG/PPG-18/18 Dimethicone and of PEG-10 Dimethicone, preferably a mixture of PEG/PPG-18/18 Dimethicone, of dimethicone and of PEG-10 Dimethicone.

19. The composition as claimed in any one of the preceding claims, where the linear polyoxyalkylenated polydimethylmethylsiloxane(s) are present at concentrations ranging from 0.1% to 10% by weight, more preferentially ranging from 0.5% to 7% by weight and more preferentially still ranging from 1% to 4% by weight, with respect to the total weight of the composition.

20. The composition as claimed in any one of the preceding claims, where the vitamin B₃ is niacinamide.

21. The composition as claimed in any one of the preceding claims, where the vitamin B₃ is present in concentrations as active material ranging from 0.01% to 20% by weight, better still from 0.1% to 10% by weight, more preferably still from 0.5% to 5% by weight, with respect to the total weight of the composition.

22. The composition as claimed in any one of the preceding claims, additionally comprising at least one pigment preferably chosen from titanium dioxides and/or iron oxides, in particular coated with a hydrophobic surface treatment agent, especially with an N-acylated amino acid and/or one of its salts, in particular with a glutamic acid derivative and/or one of its salts, especially a stearoyl glutamate, such as, for example, aluminum stearoyl glutamate.

23. The composition as claimed in claim 19, comprising at least 5% by weight of pigment(s), more preferentially from 5% to 40% by weight of pigment(s), in particular from 10% to 30% by weight, and more particularly from 10% to 20% by weight of pigment(s), with respect to the total weight of said composition.

24. The composition as claimed in any one of the preceding claims, comprising at least one nonemulsifying organopolysiloxane elastomer preferably in the gel form conveyed in at least one silicone oil, in particular a linear silicone oil of the dimethicone type.

25. The composition as claimed in claim 24, comprising a content, as active material, of nonemulsifying organopolysiloxane elastomer varying from 0.1% to 10% by weight, preferably from 0.5% to 5% by weight, with respect to the weight of the composition.

26. The composition as claimed in any one of the preceding claims, additionally comprising silica particles chosen from hydrophobic silica aerogel particles, silica particles other than the preceding ones, and their mixtures.

27. The composition as claimed in any one of the preceding claims, which is provided in the form of a foundation.
